SEQUENCE LISTING (104 sequences)

SEQ ID NO: 1 - protein, 108 aa, Artificial Sequence
  Synthetic
EVQLLESGGGLVQPGGSLRSCEASGFNAMTWVRQPPGKGLEWVSSIDSWTDAVKGRFAISQDNAKNTVYLQMNSLKPEDTAMYYCALSKCYTRVYDYWGQGTQVTVSS

SEQ ID NO: 2 - protein, 103 aa, Artificial Sequence
  Synthetic
EVQLQESGGGLVQPGGSLRLSCAASGFIYMVWVRQAPGKGLEWLSGIKTERDGVKGRFTIPRDNAKNTLYLQMNNLKSEDTALYYCATEENDWGQGTQVTVSS

SEQ ID NO: 3 - protein, 117 aa, Artificial Sequence
  Synthetic
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 4 - protein, 121 aa, Artificial Sequence
  Synthetic
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 5 - protein, 119 aa, Artificial Sequence
  Synthetic
QVQLVESGGGLVQPGGSLRLSCAASGFTFSSHAMTWVRQAPGKGLEWVAAISGSGDFTHYADSVKGRFTISRDNSKNTVSLQMNNLRAEDTAVYYCAKDEDGGSLLGYRGQGTLVTVSS

SEQ ID NO: 6 - protein, 119 aa, Artificial Sequence
  Synthetic
EVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSS

SEQ ID NO: 7 - protein, 245 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 8 - protein, 248 aa, Artificial Sequence
  Synthetic
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPLTFGAGTKLEIKGGGGSGGGGSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSS

SEQ ID NO: 9 - protein, 248 aa, Artificial Sequence
  Synthetic
DVVMTQSPSSIPVTLGESVSISCRSSKSLQNVNGNTYLYWFQQRPGQSPQLLIYRMSNLNSGVPDRFSGSGSGTDFTLRISGVEPEDVGVYYCMQHLEYPITFGAGTKLEIKGGGGSGGGGSGGGGSQVQLVQSGPELIKPGGSVKMSCKASGYTFTSYVMHWVRQKPGQGLEWIGYINPYNDGTKYNEKFKGRATLTSDKSSSTAYMELSSLRSEDSAVYYCARGTYYYGSRVFDYWGQGTTVTVSS

SEQ ID NO: 10 - protein, 246 aa, Artificial Sequence
  Synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 11 - protein, 372 aa, Artificial Sequence
  Synthetic
EVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGGGGSPAGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 12 - protein, 371 aa, Artificial Sequence
  Synthetic
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 13 - protein, 117 aa, Artificial Sequence
  Synthetic
QVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 14 - protein, 108 aa, Artificial Sequence
  Synthetic
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 15 - protein, 240 aa, Artificial Sequence
  Synthetic
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 16 - protein, 123 aa, Artificial Sequence
  Synthetic
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSS

SEQ ID NO: 17 - protein, 111 aa, Artificial Sequence
  Synthetic
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVL

SEQ ID NO: 18 - protein, 249 aa, Artificial Sequence
  Synthetic
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVLGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSS

SEQ ID NO: 19 - protein, 117 aa, Artificial Sequence
  Synthetic
EVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 20 - protein, 108 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIK

SEQ ID NO: 21 - protein, 240 aa, Artificial Sequence
  Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 22 - protein, 116 aa, Artificial Sequence
  Synthetic
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 23 - protein, 107 aa, Artificial Sequence
  Synthetic
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 24 - protein, 119 aa, Artificial Sequence
  Synthetic
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 25 - protein, 106 aa, Artificial Sequence
  Synthetic
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 26 - protein, 119 aa, Artificial Sequence [sequence omitted]

SEQ ID NO: 27 - protein, 106 aa, Artificial Sequence [sequence omitted]

SEQ ID NO: 28 - protein, 119 aa, Artificial Sequence [sequence omitted]

SEQ ID NO: 29 - protein, 107 aa, Artificial Sequence [sequence omitted]

SEQ ID NO: 30 - protein, 120 aa, Artificial Sequence
  Synthetic
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 31 - protein, 110 aa, Artificial Sequence
  Synthetic
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 32 - protein, 118 aa, Artificial Sequence
  Synthetic
EVQLVESGGGLVQPGGSLRLSCAASGFNFNDYFMNWVRQAPGKGLEWVAQMRNKNYQYGTYYAESLEGRFTISRDDSKNSLYLQMNSLKTEDTAVYYCARESYYGFTSYWGQGTLVTV

SEQ ID NO: 33 - protein, 107 aa, Artificial Sequence [sequence omitted]

SEQ ID NO: 34 - protein, 238 aa, Artificial Sequence
  Synthetic
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKGGGGSGGGGSGGGGSEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 35 - protein, 240 aa, Artificial Sequence
  Synthetic
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKGGGGSGGGGSGGGGSEVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 36 - protein, 240 aa, Artificial Sequence
  Synthetic
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKGGGGSGGGGSGGGGSEVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSS

SEQ ID NO: 37 - protein, 246 aa, Artificial Sequence
  Synthetic
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGRASGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIK

SEQ ID NO: 38 - protein, 24 aa, Artificial Sequence
  Synthetic
IYIWAPLAGTCGVLLLSLVITLYC

SEQ ID NO: 39 - protein, 42 aa, Artificial Sequence
  Synthetic
KRGRKKLLYIFKQPFMRPVQTTQEEDGCSCRFPEEEEGGCEL

SEQ ID NO: 40 - protein, 94 aa, Artificial Sequence
  Synthetic
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 41 - protein, 4 aa, Artificial Sequence
  Synthetic
YRHQ

SEQ ID NO: 42 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 43 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 691 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSPAGEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 45 - protein, 21 aa, Artificial Sequence
  Synthetic
MALPVTALLLPLALLLHAARP

SEQ ID NO: 46 - protein, 22 aa, Artificial Sequence
  Synthetic
MKYLLPTAAAGLLLLAAQPAMA

SEQ ID NO: 47 - protein, 17 aa, Artificial Sequence
  Synthetic
MGVKVLFALICIAVAEA

SEQ ID NO: 48 - protein, 18 aa, Artificial Sequence
  Synthetic
MKWVTFISLLFLFSSAYS

SEQ ID NO: 49 - protein, 19 aa, Artificial Sequence
  Synthetic
MKWVTFISLLFLFSSSSRA

SEQ ID NO: 50 - protein, 20 aa, Artificial Sequence
  Synthetic
MRRMQLLLLIALSLALVTNS

SEQ ID NO: 51 - protein, 26 aa, Artificial Sequence
  Synthetic
MATGSRTSLLLAFGLLCLPWLQEGSA

SEQ ID NO: 52 - protein, 7 aa, Artificial Sequence
  Synthetic
MALETIC

SEQ ID NO: 53 - protein, 45 aa, Artificial Sequence
  Synthetic
TTTPAPRPPTPAPTIASQPLSLRPEACRPAAGGAVHTRGLDFACD

SEQ ID NO: 54 - protein, 152 aa, Artificial Sequence
  Synthetic
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 55 - protein, 178 aa, Artificial Sequence
  Synthetic
MATGSRTSLLLAFGLLCLPWLQEGSARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 56 - protein, 159 aa, Artificial Sequence
  Synthetic
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 57 - protein, 8 aa, Artificial Sequence
  Synthetic
GGGGSPAG

SEQ ID NO: 58 - protein, 136 aa, Artificial Sequence
  Synthetic
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 59 - protein, 411 aa, Artificial Sequence
  Synthetic
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPGGGGSGGGGSGGGGSGGGGSPAGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 60 - protein, 389 aa, Artificial Sequence
  Synthetic
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSPAGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 61 - protein, 280 aa, Artificial Sequence
  Synthetic
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPGGGGSGGGGSGGGGSGGGGSQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 62 - protein, 261 aa, Artificial Sequence
  Synthetic
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSPAGQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 63 - protein, 714 aa, Artificial Sequence
  Synthetic
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSPAGEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 64 - protein, 693 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSPAGEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 65 - protein, 713 aa, Artificial Sequence
  Synthetic
MALPVTALLLPLALLLHAARPQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 66 - protein, 692 aa, Artificial Sequence
  Synthetic
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 67 - protein, 714 aa, Artificial Sequence
  Synthetic
MALPVTALLLPLALLLHAARPEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGGGGSPAGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 68 - protein, 693 aa, Artificial Sequence
  Synthetic
EVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGGGGSPAGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 69 - protein, 713 aa, Artificial Sequence
  Synthetic
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSPAGQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 70 - protein, 692 aa, Artificial Sequence
  Synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSPAGQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPRSG

SEQ ID NO: 71 - protein, 372 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSPAGEVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSS

SEQ ID NO: 72 - protein, 371 aa, Artificial Sequence
  Synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSPAGQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 73 - protein, 27 aa, Artificial Sequence
  Synthetic
SGGGSDPGGGGSGGGGSGGGGSGGGGS

SEQ ID NO: 74 - protein, 5 aa, Artificial Sequence
  Synthetic
EAAAK

SEQ ID NO: 75 - protein, 5 aa, Artificial Sequence
  Synthetic
GGGGS

SEQ ID NO: 76 - protein, 15 aa, Artificial Sequence
  Synthetic
GGGGSGGGGSGGGGS

SEQ ID NO: 77 - protein, 20 aa, Artificial Sequence
  Synthetic
GGGGSGGGGSGGGGSGGGGS

SEQ ID NO: 78 - protein, 690 aa, Artificial Sequence
  Synthetic
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 79 - protein, 691 aa, Artificial Sequence
  Synthetic
EVQLLESGGGLIQPGGSLRLSCAASGFTFSSHAMTWVRQAPGKGLEWVSAISGSGDYTHYADSVKGRFTISRDNSKNTVYLQMNSLRAEDSAVYYCAKDEDGGSLLGHRGQGTLVTVSSGGGGSPAGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 80 - protein, 690 aa, Artificial Sequence
  Synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSPAGQVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSSGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 81 - DNA, 23 bp, Artificial Sequence
  Synthetic
gaaatatacaagttatatcttgg

SEQ ID NO: 82 - RNA, 20 nt, Artificial Sequence
  Synthetic
gaaauauacaaguuauaucu

SEQ ID NO: 83 - RNA, 100 nt, Artificial Sequence
  Synthetic
gaaauauacaaguuauaucuguuuuagagcuagaaauagcaaguuaaaauaaggcuaguccguuaucaacuugaaaaaguggcaccgagucggugcuuuu

SEQ ID NO: 84 - DNA, 22 bp, Artificial Sequence
  Synthetic
tttcagctctgcatcgttttgg

SEQ ID NO: 85 - RNA, 19 nt, Artificial Sequence
  Synthetic
uuucagcucugcaucguuu

SEQ ID NO: 86 - RNA, 100 nt, Artificial Sequence
  Synthetic
guuucagcucugcaucguuuguuuuagagcuagaaauagcaaguuaaaauaaggcuaguccguuaucaacuugaaaaaguggcaccgagucggugcuuuu

SEQ ID NO: 87 - DNA, 22 bp, Artificial Sequence
  Synthetic
ttcagctctgcatcgttttggg

SEQ ID NO: 88 - RNA, 19 nt, Artificial Sequence
  Synthetic
uucagcucugcaucguuuu

SEQ ID NO: 89 - RNA, 100 nt, Artificial Sequence
  Synthetic
guucagcucugcaucguuuuguuuuagagcuagaaauagcaaguuaaaauaaggcuaguccguuaucaacuugaaaaaguggcaccgagucggugcuuuu

SEQ ID NO: 90 - DNA, 22 bp, Artificial Sequence
  Synthetic
gcatcgttttgggttctcttgg

SEQ ID NO: 91 - RNA, 19 nt, Artificial Sequence
  Synthetic
gcaucguuuuggguucucu

SEQ ID NO: 92 - RNA, 99 nt, Artificial Sequence
  Synthetic
gcaucguuuuggguucucuguuuuagagcuagaaauagcaaguuaaaauaaggcuaguccguuaucaacuugaaaaaguggcaccgagucggugcuuuu

SEQ ID NO: 93 - DNA, 22 bp, Artificial Sequence
  Synthetic
tctcttggctgttactgccagg

SEQ ID NO: 94 - RNA, 19 nt, Artificial Sequence
  Synthetic
ucucuuggcuguuacugcc

SEQ ID NO: 95 - RNA, 100 nt, Artificial Sequence
  Synthetic
gucucuuggcuguuacugccguuuuagagcuagaaauagcaaguuaaaauaaggcuaguccguuaucaacuugaaaaaguggcaccgagucggugcuuuu

SEQ ID NO: 96 - DNA, 22 bp, Artificial Sequence
  Synthetic
ttcttttacatatgggtcctgg

SEQ ID NO: 97 - RNA, 19 nt, Artificial Sequence
  Synthetic
uucuuuuacauaugggucc

SEQ ID NO: 98 - RNA, 100 nt, Artificial Sequence
  Synthetic
guucuuuuacauauggguccguuuuagagcuagaaauagcaaguuaaaauaaggcuaguccguuaucaacuugaaaaaguggcaccgagucggugcuuuu

SEQ ID NO: 99 - DNA, 22 bp, Artificial Sequence
  Synthetic
ttctgcttcttttacatatggg

SEQ ID NO: 100 - RNA, 19 nt, Artificial Sequence
  Synthetic
uucugcuucuuuuacauau

SEQ ID NO: 101 - RNA, 100 nt, Artificial Sequence
  Synthetic
guucugcuucuuuuacauauguuuuagagcuagaaauagcaaguuaaaauaaggcuaguccguuaucaacuugaaaaaguggcaccgagucggugcuuuu

SEQ ID NO: 102 - DNA, 22 bp, Artificial Sequence
  Synthetic
tttctgcttcttttacatatgg

SEQ ID NO: 103 - RNA, 19 nt, Artificial Sequence
  Synthetic
uuucugcuucuuuuacaua

SEQ ID NO: 104 - RNA, 100 nt, Artificial Sequence
  Synthetic
guuucugcuucuuuuacauaguuuuagagcuagaaauagcaaguuaaaauaaggcuaguccguuaucaacuugaaaaaguggcaccgagucggugcuuuu

(19) United States
(12) Patent Application Publication
(10) Pub. No.: US 2024/0245772 A1
(43) Pub. Date: Jul. 25, 2024

(54) BI-SPECIFIC CHIMERIC ANTIGEN RECEPTORS AND GENETICALLY ENGINEERED IMMUNE CELLS EXPRESSING SUCH

(71) Applicant: Celledit LLC, Worcester, MA (US)

(72) Inventor: Biliang Hu, Worcester, MA (US)

(73) Assignee: Celledit LLC, WORCESTER, MA (US)

(21) Appl. No.: 18/561,307

(22) PCT Filed: May 18, 2022
(86) PCT No.: PCT/US2022/029915

§ 371 Date: Nov. 16, 2023

Related U.S. Application Data
(60) Provisional application No. 63/190,480, filed on May 19, 2021.

(51) Int. Cl. A61K 39/00; A61P 35/00; C07K 14/545; C07K 14/725; C07K 16/24; C07K 16/28; C12N 5/0783; C12N 15/86

(52) U.S. Cl. CPC A61K 39/464417; A61K 39/4611; A61K 39/4631; A61K 39/464412; A61P 35/00; C07K 14/545; C07K 14/7051; C07K 16/248; C07K 16/2803; C07K 16/2866; C07K 16/2878; C12N 5/0636; C12N 15/86; A61K 2039/572; C07K 2317/31; C07K 2317/622; C07K 2319/02; C12N 2740/15043

(57) ABSTRACT

A bi-specific chimeric antigen receptor (bi-specific CAR) comprising a single chain variable fragment (scFv) and a single variable domain (VHH) in the extracellular antigen binding domain, wherein the scFv and VHH bind tumor associated antigens. Also provided herein are genetically engineered immune cells expressing such bi-specific CAR and therapeutic uses of the genetically engineered immune cells.

### CROSS-REFERENCE TO RELATED APPLICATIONS

This application claims the benefit of priority to U.S. Provisional Patent Application No. 63/190,480, filed May 19, 2021, which is hereby incorporated by reference in its entirety.

### BACKGROUND OF THE INVENTION

Adoptive cell transfer therapy is a type of immunotherapy that involves ex vivo expansion of autologous or allogeneic immune cells and subsequent infusion into a patient. The immune cells may be modified ex vivo to specifically target malignant cells. Modifications include engineering of T cells to express chimeric antigen receptors (CARs). The promise of adoptive cell transfer therapy, such as CAR T-cell (CAR-T) therapy is often limited by toxicity (e.g., cytokine-associated toxicity). For example, adoptive cell transfer immunotherapy may trigger non-physiologic elevation of cytokine levels (cytokine release syndrome), which could lead to death of recipients (see, e.g., Morgan et al., Molecular Therapy 18(4): 843-851, 2010). In addition, modified immune cells may not expand well in patients, may not persist long in vivo, and may be susceptible to the cytotoxic environment initiated by their own activities in vivo.

It is therefore of great interest to develop approaches to improve the proliferation of these modified immune cells and reduce toxicity associated with CAR-T therapy, while maintaining or enhancing therapeutic efficacy.

### SUMMARY OF THE INVENTION

The present disclosure is based, at least in part, on the development of a bi-specific chimeric antigen receptor (CAR) comprising a bi-specific extracellular antigen binding domain that binds two separate antigens or antigen epitopes, thereby improving therapeutic efficacy of the immune cells expressing such in vivo.

In some aspects, the present disclosure provides a bi-specific chimeric antigen receptor (CAR) polypeptide, comprising: (a) a first antigen binding moiety, (b) a second antigen binding moiety, (c) a co-stimulatory signaling domain, and (d) a cytoplasmic signaling domain. The first antigen binding moiety can be a single domain antibody variable fragment such as a VHH fragment and the second antigen binding moiety can be a single chain variable fragment (scFv).

The first antigen binding moiety binds a first tumor-associated antigen, and the second antigen binding moiety binds a second tumor-associated antigen, which is different from the first tumor associated antigen. In some instances, the first and second tumor antigens are selected from 5T4, CD2, CD3, CD5, CD7, CD19, CD20, CD22, CD30, CD33, CD38, CD70, CD123, CD133, CD171, CEA, CS1, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, Claudin 18.2, and VEGFRII. In specific examples, the first tumor antigen is CD19, and the second tumor antigen is BCMA. Alternatively, the first tumor antigen is BCMA, and the second tumor antigen is CD19.

In some embodiments, the first antigen binding moiety in the bi-specific CAR polypeptide disclosed herein is a VHH fragment binding to CD19 (anti-CD19 VHH) and the second antigen binding moiety is a scFv binding to BCMA (anti-BCMA scFv). Alternatively, the first antigen binding moiety is a VHH binding to BCMA (anti-BCMA VHH) and the second antigen binding moiety is a scFv fragment binding to CD19 (anti-CD19 scFv).

In some examples, the bi-specific CAR comprises an anti-CD19 scFv, which may comprise the amino acid sequence of SEQ ID. NO: 7, 8, or 9. Alternatively or in addition, the bi-specific CAR further comprises an anti-BCMA VHH, which may comprise the amino acid sequence of SEQ ID NO: 4, 5, or 6. In specific examples, the bi-specific CAR comprises the amino acid sequence of SEQ ID NO: 11 (e.g., as the extracellular bi-specific antigen binding domain).

In other examples, the bi-specific CAR comprises an anti-CD19 VHH, which may comprise the amino acid sequence of SEQ ID NO: 1, 2, or 3. Alternatively or in addition, the bi-specific CAR further comprises an anti-BCMA scFv, which may comprise the amino acid sequence of SEQ ID NO: 10. Such a bi-specific CAR may comprise the amino acid sequence of SEQ ID NO: 11, 12, 71, or 72. In one specific example, the bi-specific CAR comprises the amino acid sequence of SEQ ID NO: 11 (e.g., as the extracellular bi-specific antigen binding domain). In another specific example, the bi-specific CAR comprises the amino acid sequence of SEQ ID NO: 12 (e.g., as the extracellular bi-specific antigen binding domain).

In other aspects, the present disclosure provides a bi-specific chimeric antigen receptor (CAR) polypeptide, comprising: (a) a first antigen binding moiety, which is a truncated fragment of APRIL that binds to BCMA; (b) a second antigen binding moiety, which is a single domain antibody variable fragment (VHH) or a single chain variable fragment (scFv) that binds a tumor associated antigen (e.g., CD19), (c) a co-stimulatory signaling domain, and (d) a cytoplasmic signaling domain.

In some instances, the truncated fragment of APRIL that binds BCMA comprises an amino acid sequence at least 90% identical to SEQ ID NO: 58. In some examples, the truncated fragment of APRIL comprises the amino acid sequence of SEQ ID NO: 58. Alternatively or in addition, the second antigen-binding moiety is an anti-CD19 scFv or an anti-CD19 VHH. In some examples, the second antigen-binding moiety is an anti-CD19 scFv, which may comprise the amino acid sequence of SEQ ID NO: 7, 8, or 9. In other examples, the second antigen-binding moiety is an anti-CD19 VHH, which may comprise the amino acid sequence of SEQ ID NO: 1, 2, or 3. In specific examples, the bi-specific CAR polypeptide may comprise the amino acid sequence of SEQ ID NO: 59, 60, 61, or 62 (e.g., as the extracellular bi-specific antigen binding domain).

Any of the bi-specific CAR polypeptides disclosed herein may further comprise a peptide linker between the first antigen binding moiety and the second antigen binding moiety. Such a peptide linker may be about 4-40 amino acids in length. In some examples, the bi-specific CAR polypeptide disclosed herein may comprise a co-stimulatory signaling domain from 4-1BB or CD28. Alternatively or in addition, the cytoplasmic signaling domain in the bi-specific CAR polypeptide may comprise a CD3z cytoplasmic signaling domain, an IL-2RB cytoplasmic signaling domain, or a combination thereof. In specific examples, the cytoplasmic signaling domain in the bi-specific CAR polypeptide comprises both the CD3□ cytoplasmic signaling domain and the IL-2Rβ cytoplasmic signaling domain. In some instances, the cytoplasmic signaling domain comprises the CD3z cytoplasmic signaling domain, which optionally comprises a STAT binding motif, e.g., at the C-terminus.

Any of the bi-specific CAR polypeptides disclosed herein may further a transmembrane domain, a hinge domain, or a combination thereof. In some instances, the transmembrane domain and/or the hinge domain can be located between the first or second antigen binding moiety and the co-stimulatory domain. In some examples, the transmembrane domain and/or the hinge domain is from CD8.

Exemplary bi-specific CAR polypeptides provided herein may comprise any of the amino acid sequence of SEQ ID NOs: 63-70.

In other aspects, the present disclosure also provides a population of genetically engineered immune cells, which expressing a bi-specific CAR polypeptide as disclosed herein. The population of genetically engineered immune cells such as T cells may further comprise one or more of the following features: (a) have one or more disrupted endogenous genes encoding one or more proinflammatory cytokines; and (b) express one or more antagonists targeting the proinflammatory cytokines. In some embodiments, the proinflammatory cytokines include interferon gamma (IFNγ), interleukin 6 (IL-6), GM-CSF, interleukin 1 (IL-1), or a combination thereof.

In some embodiments, the population of genetically engineered immune cells may comprise a disrupted endogenous interferon gamma gene, a disrupted endogenous GM-CSF gene, or a combination thereof. In some instances, the endogenous interferon gamma gene, the endogenous GM-CSF gene, or both are disrupted by a CRISPR/Cas gene editing system.

Alternatively or in addition, the genetically engineered immune cells express an IL-6 antagonist, an IFNγ antagonist, an IL-1 antagonist, or a combination thereof. In some examples, the IL-6 antagonist is an antibody specific to human IL6 (anti-IL6 antibody) or an antibody specific to human IL6R (anti-IL6R antibody). In some examples, the IFNγ antagonist is an antibody specific to human IFNγ (anti-IFNγ antibody). In some instances, the anti-IL6 antibody, the anti-IFNγ antibody, or both can be scFv antibodies.

In some examples, the genetically engineered immune cells express an anti-IFNγ scFv comprising a heavy chain variable region, which comprises the amino acid sequence of SEQ ID NO: 13, and a light chain variable region, which comprises the amino acid sequence of SEQ ID NO: 14. Such an anti-IFNγ scFv may comprise the amino acid sequence of SEQ ID NO: 15. In other examples, the genetically engineered immune cells express an anti-IFNγ scFv comprising a heavy chain variable region, which comprises the amino acid sequence of SEQ ID NO: 16, and a light chain variable region, which comprises the amino acid sequence of SEQ ID NO: 17. Such an anti-IFNγ scFv may comprise the amino acid sequence of SEQ ID NO: 18. In yet other examples, the genetically engineered immune cells express an anti-IFNγ scFv comprising a heavy chain variable region, which comprises the amino acid sequence of SEQ ID NO: 19, and a light chain variable region, which comprises the amino acid sequence of SEQ ID NO: 20. Such an anti-IFNγ scFv may comprise the amino acid sequence of SEQ ID NO: 21.

In some specific examples, the genetically engineered immune cells expressing any of the anti-IFNγ scFv antibodies disclosed herein may further express a bi-specific CAR comprising the amino acid sequence of SEQ ID NO: 63, 64, 65, or 66.

In some examples, the genetically engineered immune cells express an anti-IL6 scFv, which may comprise a heavy chain variable region, which comprises the amino acid sequence of SEQ ID NO: 24, and a light chain variable region, which comprises the amino acid sequence of SEQ ID NO: 25. In other examples, the genetically engineered immune cells express an anti-IL6 scFv, which may comprise a heavy chain variable region, which comprises the amino acid sequence of SEQ ID NO: 26, and a light chain variable region, which comprises the amino acid sequence of SEQ ID NO: 27. In yet other examples, the genetically engineered immune cells express an anti-IL6 scFv, which may comprise a heavy chain variable region, which comprises the amino acid sequence of SEQ ID NO: 30, and a light chain variable region, which comprises the amino acid sequence of SEQ ID NO: 31.

Alternatively, the genetically engineered immune cells express an anti-IL6R scFv, which may comprise a heavy chain variable region, which comprises the amino acid sequence of SEQ ID NO: 22, and a light chain variable region, which comprises the amino acid sequence of SEQ ID NO: 23. In other examples, the genetically engineered immune cells express an anti-IL6R scFv, which may comprise a heavy chain variable region, which comprises the amino acid sequence of SEQ ID NO: 28, and a light chain variable region, which comprises the amino acid sequence of SEQ ID NO: 29. In yet other examples, the genetically engineered immune cells express an anti-IL6R scFv, which may comprise a heavy chain variable region, which comprises the amino acid sequence of SEQ ID NO: 32, and a light chain variable region, which comprises the amino acid sequence of SEQ ID NO: 33.

In specific examples, the genetically engineered immune cells may express an anti-IL6 scFv or anti-IL6R scFv comprising the amino acid sequence of SEQ ID NO: 34, 35, 36, or 37.

In some examples, the genetically engineered immune cells express an IL-1 antagonist is IL-1RA, which may comprise the amino acid sequence of SEQ ID NO: 36.

The population of genetically engineered immune cells disclosed herein may comprise T cells, tumor infiltrating lymphocytes, Natural Killer (NK) cells, dendritic cells, macrographs, B cells, neutrophils, eosinophils, basophils, mast cells, myeloid-derived suppressor cells, mesenchymal stem cells, precursors thereof, or a combination thereof. In some instances, the immune cells are human immune cells. In specific examples, the human immune cells comprise human T cells.

In addition, the present disclosure provides a pharmaceutical composition, comprising the population of immune cells disclosed herein and a pharmaceutically acceptable carrier.

In yet other aspects, the present disclosure features a method for reducing or eliminating undesired cells in a subject, the method comprising administering to a subject in need thereof a therapeutically effective amount of the population of immune cells disclosed herein or the pharmaceutical composition comprising such. In some instances, the subject is a human patient having a cancer, which comprises cancer cells expressing the first tumor associated antigen, the second tumor associated antigen, or both.

In some examples, the subject is a human patient having a solid tumor or a hematological cancer. For example, the human patient may have a solid tumor, which can be breast cancer, lung cancer, pancreatic cancer, liver cancer, glioblastoma (GBM), prostate cancer, ovarian cancer, mesothelioma, colon cancer, or stomach cancer. In other examples, the human patient may have a hematological cancer, which can be leukemia, lymphoma, or multiple myeloma.

Also within the scope of the present disclosure are immune cell populations and pharmaceutical composition as described herein for use in treating a target disease as described herein (e.g., cancer), and uses of such immune cell population and pharmaceutical composition in manufacturing a medicament for use in treatment of the target disease, such as cancer.

The details of one or more embodiments of the invention are set forth in the description below. Other features or advantages of the present invention will be apparent from the following drawings and detailed description of several embodiments, and from the appended claims.

### BRIEF DESCRIPTION OF THE DRAWINGS

FIG. 1 is a schematic diagram showing exemplary designs of bi-specific chimeric antigen receptor (CAR) polypeptides having tandem arrangements of the antigen binding moieties (e.g., scFv and VHH) in the extracellular antigen-binding domain of the bi-specific CARs.

FIGS. 2A-2C include diagrams showing CAR-T cell expansion and levels of IFNγ in peripheral blood of ALL patients receiving bi-specific anti-BCMA VHH/anti-CD19 scFv CAR T cells secreting an exemplary anti-IFNγ scFv, and optionally an exemplary anti-IL6 scFv. FIG. 2A: CAR+ T cell expression from an ALL patient receiving the bi-specific anti-BCMA VHH/anti-CD19 scFv CAR T cells secreting the exemplary anti-IFNγ scFv. FIG. 2B: peripheral IFNγ level in the ALL patient. FIG. 2C: levels of IFNγ in peripheral blood of an ALL patient receiving the bi-specific anti-BCMA VHH/anti-CD19 scFv CAR T cells secreting both the exemplary anti-IFNγ scFv and the exemplary anti-IL6 scFv.

FIGS. 3A-3E include diagrams showing CAR-T cell expansion and levels of IFNγ in peripheral blood of patients diagnosed with refractory and relapsed multiple myeloma (MM) and treated with genetically engineered T cells expressing a bi-specific anti-CD19 VHH scFv/anti-BCMA scFv CAR alone, or in combination with anti-IFNγ scFv. FIGS. 3A and 3C-3D: CAR-T cell expansion in patients treated with genetically engineered T cells co-expressing the bispecific CAR and the anti-IFNγ scFv. FIG. 3B: blood levels of IFNγ in an exemplary patient treated with genetically engineered T cells co-expressing the bispecific CAR and the anti-IFNγ scFv. FIG. 3E: CAR-T cell expansion in a patient treated with genetically engineered T cells expressing the bispecific CAR but not the anti-IFNγ scFv.

FIGS. 4A-4C include diagrams showing in vitro cytotoxicity of genetically engineered T cells expressing a bi-specific anti-CD19 VHH scFv/anti-BCMA scFv CAR alone, or in combination with anti-IFNγ scFv. FIG. 4A: targeting Nalm6 cells. FIG. 4B: targeting MM1S cells. FIG. 4C: targeting RPMI 8226 cells.

### DETAILED DESCRIPTION OF THE INVENTION

Adoptive cell transfer immunotherapy relies on immune cell activation and cytokine secretion to eliminate disease cells such as cancer cells. However, CAR-T cells do not always expand or activate well in patients.

The present disclosure aims to overcome limitations associated with current adoptive CAR-T therapy by, e.g., the development of a bi-specific chimeric antigen receptor (CAR) targeting multiple tumor-associated antigens or multiple parts of a tumor associated antigen, thereby improving therapeutic efficacy in vivo. In some instances, the multiple antigen-binding moieties in the bi-specific CAR disclosed herein may be in a combination of single-domain antibody format (e.g., VHH) and single-chain variable fragment (scFv) format.

It was observed that bi-specific CAR including the scFv-scFv tandem format exhibited CAR expression problems in some instances, which may be caused by the interference between the two scFv binding moieties. Without being bound by theory, the VHH/scFv bi-specific CAR format is designed to solve this potential CAR expression problem. The exemplary bi-specific CARs in the VHH/scFv format tested so far all exhibited satisfactory expression in immune cells. Genetically engineered immune cells (e.g., T cells) expressing the bi-specific CAR disclosed herein may comprise additional genetic modifications, for example, engineered to express an antagonist of a proinflammatory cytokine, engineered to disrupt an endogenous gene of a proinflammatory cytokine, or a combination thereof.

### I. Bispecific Chimeric Antigen Receptor

In some aspects, the present disclosure provides a bi-specific chimeric antigen receptor (CAR) capable of binding to two different tumor-associated antigens or two different antigenic epitopes (which may be in the same antigen) of tumor-associated antigen(s).

A CAR is an artificial (non-naturally occurring) receptor having binding specificity to a target antigen of interest (e.g., a tumor cell antigen) and capable of triggering immune responses in immune cells expression such upon binding to the target antigen. A CAR often comprises an extracellular antigen-binding domain fused to at least an intracellular signaling domain. Cartellieri et al., J Biomed Biotechnol 2010:956304, 2010. The bi-specific CAR disclosed herein comprise two antigen-binding moieties (i.e., a first antigen-binding moiety and a second antigen-binding moiety) having specificity to different target antigens or different antigenic epitopes. In some instances, the bi-specific CAR disclosed herein may be a single polypeptide comprising the two antigen-binding moieties as the extracellular domain and an intracellular domain, which may comprise one or more signaling domains, e.g., a co-stimulatory signaling domain, a cytoplasmic signaling domain, or a combination thereof. The extracellular domain and the intracellular domain may be linked via a hinge domain, a transmembrane domain, or a combination thereof.

In some examples, a flexible peptide linker, e.g., a G/S rich linker, may be used to connect two adjacent functional domains, for example, the two antigen-binding moieties. For example, the G/S rich linker may comprise the motif of (G₄S)ₙ, in which n is 1, 2, 3, 4, 5, or 6. Exemplary G/S rich linkers include G₄S (SEQ ID NO: 75), (G₄S)₃ (SEQ ID NO: 76), or and (G₄S)₄ (SEQ ID NO: 77). In another example, the flexible peptide linker may comprise the motif of EAAAK (SEQ ID NO: 74). Such a peptide linker may contain one or more copies of the motif, e.g., 1, 2, 3, 4, 5, or 6 copies of the motif.

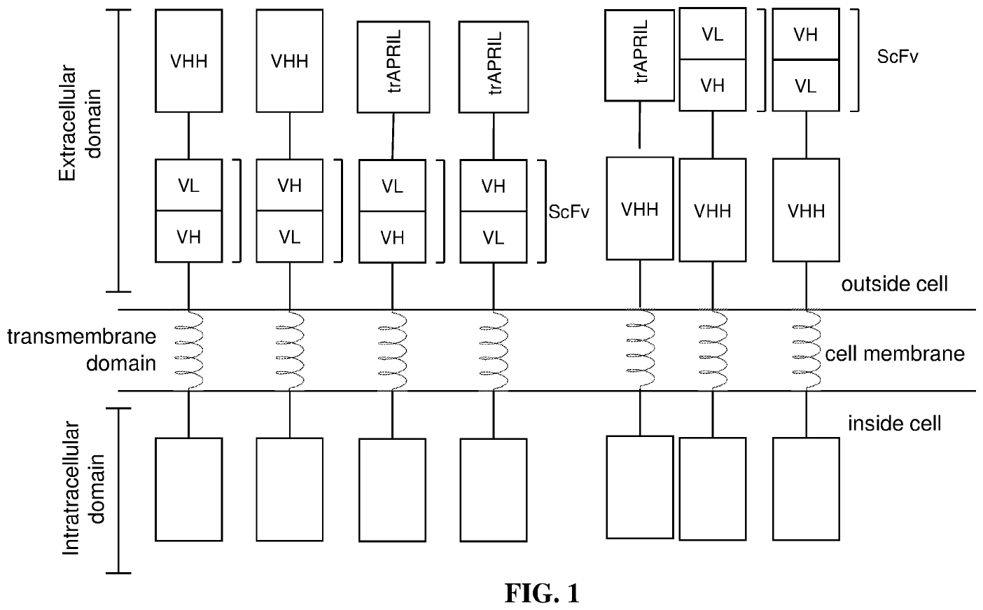

Exemplary designs of the bi-specific CAR disclosed herein can be found in FIG. 1.

### (a) Bi-Specific Extracellular Antigen Binding Domain

The extracellular antigen-binding domain of the bi-specific CAR polypeptide disclosed herein is specific to two antigens of interest (e.g., a pathologic antigen such as a tumor-associated antigen, also known as a cancer antigen) or two antigenic epitopes. As used herein, tumor-associated antigens (TAA) are antigens that exhibit elevated levels on tumor cells or a specific type of tumor cells as relative to non-tumor cells or other types of tumor cells.

The extracellular antigen-binding domain comprises a first antigen-binding domain and a second antigen-binding domain capable of binding to the two antigens of interest (e.g., two tumor-associated antigens) or the two antigenic epitopes of an antigen of interest. Antigens of interest can also be any natural molecules expressed on cells that has been identified as a promising immunotherapy target antigen for various types of cancers.

In some embodiment, the first antigen-binding domain of the bi-specific CAR polypeptide described herein can be in a single-domain antibody format, for example, a heavy-chain only antibody fragment (VHH), and the second antigen-binding domain can be in a single-chain variable fragment (scFv) format.

A single-domain antibody such as VHH is a type of antibody containing a single monomeric variable antibody domain. Such antibodies may be derived from the Alpaca heavy chain IgG antibody. Alternatively, VHH antibodies capable of binding to a specific target antigen may be isolated via a conventional method, for example, antibody library screening.

A scFv fragment contains a heavy chain variable region (VH) and a light chain variable region (VL) linked by a flexible peptide linker. In some examples, the scFv may be in the VH to VL orientation (from N-terminus to C-terminus). Alternatively, the scFv may be in the VL to VL orientation (from N-terminus to C-terminus). The flexible peptide linker for use to connect the VH and VL domains of a scFv fragment (or any two adjacent functional domains in the bi-specific CAR polypeptide disclosed herein) may be a G/S rich peptide linker, which is commonly used in the art in fusion polypeptides. Exemplary peptide linkers are provided in Sequence Table 2 below.

The VHH and scFv may be connected via a flexible peptide linker such as a G/S peptide linker, which is commonly used in the art for connecting two functional domains. In some instances, the extracellular domain may be in the VHH to scFv orientation (from N-terminus to C-terminus). Alternatively, the extracellular domain may be in the scFv to VHH orientation (from N-terminus to C-terminus). See exemplary arrangements shown in FIG. 1.

In some embodiments, the first antigen-binding domain and the second antigen-binding domain may bind to two tumor-associated antigens. Non-limiting examples of tumor associated antigens include 5T4, CD2, CD3, CD5, CD7, CD19, CD20, CD22, CD30, CD33, CD38, CD70, CD123, CD133, CD171, CEA, CS1, BCMA, BAFF-R, seprase (also known as FAP), PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, Claudin 18.2, and VEGFRII.

In other examples, one of the target tumor antigens is FAP, which is a surface-expressed proteolytic enzyme that expressed on cancer-associated fibroblasts (CAFs). FAP is viewed as a major component of the stromal microenvironment of carcinomas such as prostate, lung and pancreatic cancer, and mesothelioma. Moreover, FAP was consistently overexpressed in a large proportion of patient tumors and patient-derived glioblastoma cultures compared to normal tissue.

In some embodiments, the extracellular antigen-binding domain of the bi-specific CAR targets CD19 and B-cell maturation antigen (BCMA). In some examples, the extracellular antigen-binding domain comprises an anti-CD19 antigen binding domain in VHH format (anti-CD19 VHH). Examples of anti-CD19 VHH fragments are provided in Sequence Table 1 (SEQ ID NOs: 1-3). See, e.g., S. R. Banihashemi, et al., Iran J Basic Med Sci, 21(5):455-464, 2018), and CN 1053848258, the relevant disclosures of which are incorporated by reference for the subject matter and purpose referenced herein. Alternatively, the extracellular antigen-binding domain comprises an anti-CD19 antigen binding domain in scFv format (anti-CD19 scFv). Examples of anti-CD19 scFv are also provided in Sequence Table 1 (SEQ ID NOs: 7-9, 71). See also WO 2020/135335, the content is incorporated herein by reference in its entirety. In some instances, the anti-CD19 VHH or anti-CD19 scFv may be derived from the exemplary anti-CD19 VHH or exemplary anti-CD19 scFv provided in Sequence Table 1, for example, having the same heavy chain and light chain complementary determining regions (CDRs). Heavy and light chain CDRs of the exemplary antibodies listed in Sequence Table 1, determined based on the Kabat definition, are in boldface and underlined.

The extracellular-binding domain of the bi-specific CAR targeting CD19 and BCMA may comprise an anti-BCMA antigen binding domain in VHH format (anti-BCMA VHH). Examples of anti-BCMA VHH fragments are provided in Sequence Table 1 (SEQ ID NOS: 4-6). See also WO2018/237037, the relevant disclosures of which are incorporated by reference for the subject matter and purpose referenced herein. Alternatively, the extracellular antigen-binding domain comprises an anti-BCMA antigen binding domain in scFv format (anti-BCMA scFv). Examples of anti-BCMA scFv are also provided in Sequence Table 1 (SEQ ID NOs: 10-12, 72). In some instances, the anti-BCMA VHH or anti-BCMA scFv may be derived from any of the exemplary anti-BCMA VHH or exemplary anti-BCMA scFv provided in Sequence Table 1, for example, having the same heavy chain and light chain complementary determining regions (CDRs). Heavy and light chain CDRs of the exemplary antibodies listed in Sequence Table 1, determined based on the Kabat definition, are in boldface and underlined.

The anti-CD19/anti-BCMA bi-specific CAR polypeptides described herein may comprise an anti-CD19 VHH binding moiety and an anti-BCMA scFv binding moiety, which may be in any suitable orientation, for example, anti-CD19 VHH/anti-BCMA scFv (N-terminus to C-terminus) or anti-BCMA scFv/anti-CD19 VHH (N-terminus to C-terminus). The anti-CD19 VHH and anti-BCMA scFv fragments may be linked via a flexible peptide linker, e.g., those provided in Sequence Table 1 and Sequence Table 2. Alternatively, the anti-CD19/anti-BCMA bi-specific CAR polypeptides described herein may comprise an anti-BCMA VHH binding moiety and an anti-CD19 scFv binding moiety, which may be in any suitable orientation, for example, anti-BCMA VHH/anti-CD19 scFv (N-terminus to C-terminus) or anti-CD19 scFv/anti-BCMA VHH (N-terminus to C-terminus). The anti-BCMA VHH and anti-CD19 scFv fragments may be linked via a flexible peptide linker, e.g., those provided in Sequence Table 1 and Sequence Table 2.

In some examples, the anti-CD19/anti-BCMA bi-specific CAR polypeptide described herein comprises (a) an anti-CD19 scFv, which comprises the amino acid sequence of SEQ. ID. NO: 7, 8, or 9, and (b) an anti-BCMA VHH comprising the amino acid sequence of SEQ. ID. NO: 4, 5, or 6.

In some examples, the anti-CD19/anti-BCMA bi-specific CAR polypeptide described herein comprises (a) an anti-CD19 VHH, which comprises the amino acid sequence of SEQ. ID. NO: 1, 2, or 3, and (b) an anti-BCMA scFv, which comprises the amino acid sequence of SEQ. ID. NO: 10.

Exemplary extracellular domains of a bi-specific CAR as disclosed herein, which targets both CD19 and BCMA, comprise the amino acid sequence of any one of SEQ ID NOs: 11, 12, 71, and 72 provided in Sequence Table 1.

In some embodiments, the anti-CD19/anti-BCMA bi-specific CAR polypeptide may comprise (a) a truncated APRIL fragment that binds BCMA (e.g., residues 116 to 250 of the canonical sequence for APRIL (Uniprot 075888), Lee, L. et al., 2018, Blood, 131(7): 746-758), and (b) an antigen-binding moiety that binds CD19, e.g., in VHH or scFv format such as any of the anti-CD19 VHH or anti-CD19 scFv disclosed herein (see Sequence Table 1). APRIL (APRoliferation-Inducing Ligand) is a natural high-affinity ligand for BCMA and transmembrane activator and calcium-modulator and cyclophilin ligand (TACI). APRIL is also known as TNFSF13. The amino terminus of APRIL binds proteoglycans but is not involved in the interaction with BCMA or TACI. In some instances, a truncated APRIL fragment (trAPRIL) may comprise (e.g., consisting of) residues 116 to 250 of the naturally-occurring human APRIL for binding to BCMA but having no the proteoglycan binding activity. In one instance, the trAPRIL lacks the N-terminal 115 amino acids from the wild-type APRIL molecule. See U.S. Pat. No. 10,160,794, the relevant disclosures of which are incorporated by reference for the purpose and subject matter referenced herein. As one example, the trAPRIL for making the bi-specific CAR can be set forth as SEQ ID NO:58. Alternatively, the trAPRIL fragment may be at least 85%, 88%, 90%, 92%, 95%, 97%, 99% identity to SEQ ID NO: 58 and binds BCMA. BCMA binding can be determined by any method known in the art, e.g., as described in U.S. Pat. No. 10,160,794.

In some examples, the anti-CD19 moiety may be an anti-CD19 scFv, e.g., comprising the amino acid sequence of SEQ ID NO: 7, 8, or 9. Alternatively, the anti-CD19 moiety can be an anti-CD19 VHH, e.g., comprising the amino acid sequence of SEQ ID NO: 1, 2, or 3. The anti-CD19 moiety may be linked to the trAPRIL via a flexible peptide linker, e.g., those disclosed herein (e.g., SEQ ID NO: 57 or 73). In some instances, the anti-CD19 moiety can be located at the N-terminal portion relative to the trAPRIL. Alternatively, the trAPRIL can be located at the N-terminal portion relative to the anti-CD19 moiety. Examples of trAPRIL-containing bi-specific extracellular domains include SEQ ID NOs: 59, 60, 61, and 62.

### (b) Intracellular Signaling Domains

Any of the bi-specific CAR polypeptides disclosed herein may further comprise a co-stimulatory domain. Non-limiting sources for co-stimulatory domains include OX40, CD70, CD27, CD28, CD5, ICAM-1, LFA-1 (CD11a/CD18), ICOS (CD278), DAP10, and DAP12. Hence, the CAR may have a co-stimulatory domain derived from 4-1BB, OX40, CD70, CD27, CD28, CD5, ICAM-1, LFA-1 (CD11a/CD18), ICOS (CD278), DAP10, and DAP12 or any combination thereof. In some examples, the bi-specific CAR may comprise a co-stimulatory domain from co-stimulatory receptor 4-1BB (aka CD137), for example, from the human 4-1BB. One exemplary of a 4-1BB co-stimulatory signaling domain comprises (e.g., consists of) the amino acid sequence SEQ ID NO: 39.

Alternatively or in addition, the bi-specific CAR polypeptide may further comprise a cytoplasmic signaling domain comprising an ITAM such as a CD3ζ signaling domain. Exemplary CD3ζ signaling domains include, but are not limited to, fragments comprising (e.g., consisting of) SEQ ID NO: 43. In some instances, a CD3ζ signaling domain may be modified to insert a STAT binding motif, e.g., linked to its C-terminal portion. The STAT3 binding motif may have the amino acid sequence YX₁X₂Q, where X₁ and X₂ are each independently an amino acid. In particular, the YX₁X₂Q motif may be YRHQ (SEQ. ID. NO: 41). In some examples, the fragment in the CAR construct containing the CD3ζ signaling domain and the STAT3 binding motif may comprise (e.g., consist of) the amino acid sequence of SEQ ID NO: 42.

In some instances, the bi-specific CAR polypeptide disclosed herein may further comprise an IL-2RB signaling domain, which optionally may be in combination with an ITAM-containing cytoplasmic signaling domain, such as a CD3□ signaling domain, an additional co-stimulatory domain such as that from 4-1BB, or a combination thereof. Without being bound by theory, the presence of the IL2RB signaling domain may significantly improve persistence in vivo of the CAR-T cells expressing the bi-specific CAR polypeptide comprising such. IL2RB is the β chain of the interleukin-2 receptor (IL-2R). An IL-2Rβ signaling domain refers to the fragment in an IL2Rβ polypeptide (e.g., of a suitable species such as human) that is capable of triggering the signaling pathway mediated by the IL-2/IL-2R interaction. IL-2Rβ polypeptides and the signaling domains therein are known in the art. For example, the human IL-2Rβ polypeptide is provided in GENBANK accession number NP_000869.1 (the contents of which are incorporated herein by reference). IL-2Rβ polypeptides from other species can be obtained from publicly available gene databases such as GENBANK.

In some examples, the IL2Rβ signaling domain used in the bi-specific CAR polypeptide disclosed herein comprise an amino acid sequence at least 80% (e.g., at least 85%, 90%, 95%, 98% or above) identical to the amino acid sequence of SEQ ID NO: 40. In one example, the IL2Rβ signaling domain comprises (e.g., consists of) SEQ ID NO: 40.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

### (c) Other CAR Components

Any of the bi-specific CAR polypeptides disclosed herein may further comprise a transmembrane domain (TMD), a hinge domain, or both. In some examples, the TMD may be located between the extracellular antigen binding domain and the intracellular signaling domain. See FIG. 1. Alternatively or in addition, the hinge domain may be located between the extracellular antigen-binding domain and the TMD, between the TMD and the intracellular signaling domain, or within the intracellular signaling domain when the intracellular signaling domain comprises a combination of one or more co-stimulatory signaling domain and/or a cytoplasmic signaling domain. Any TMD and/or hinge domains commonly used in bi-specific CAR polypeptide construction can be used here. See U.S. Pat. No. 10,160,794.

In some examples, the TMD may be obtained from a suitable cell-surface receptor, such as the cell surface receptor of the alpha, beta or zeta chain of the T-cell receptor, CD28, CD3 epsilon, CD3 delta, CD3 gamma, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR). In some examples, the hinge domain may be of CD28, CD8, an IgD or an IgG, such as IgG1 or IgG4. See U.S. Pat. No. 10,160,794. In one example, the TMD may be of human CD8a, e.g., comprising or consisting of the amino acid sequence of SEQ. ID. NO:38.

In some examples, the bi-specific CAR may also comprise a hinge domain, which may be linked to the C-terminus of the bi-specific extracellular antigen binding domain and the N-terminus of the transmembrane domain. Suitable hinge domains can be derived from CD28, CD8, IgD or an IgG; such as IgG1 and IgG4. In one example, the hinge domain may be of human CD8, e.g., comprising or consisting of the amino acid sequence of SEQ. ID. NO:53. In some instances, the TMD and hinge domain may be connected via a flexible peptide linker such as those disclosed herein.

Any component for use in constructing the bi-specific CAR polypeptides may be a fragment of a naturally-occurring protein (e.g., a cellular receptor such as an immune cell receptor such as those disclosed herein). Alternatively, the CAR component may be a variant of a wild-type counterpart, which may share at least 90% sequence identity to the wild-type counterpart and maintain substantially the same bioactivity. In some instances, the variant may contain up to 15 (e.g., up to 12, 10, 8, 6, 5, 4, 3, 2, or 1) amino acid residue substitutions relative to the wild-type counterpart. In some examples, the one or more amino acid residue substitutions are conservative amino acid residue substitutions.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: ((a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

### (d) Exemplary Bi-Specific CAR Polypeptides

Exemplary bi-specific CAR polypeptides disclosed herein may comprise, from N-terminus to C-terminus, a first antigen-binding moiety, a flexible peptide linker (e.g., SEQ ID NO: 57), a second antigen-binding moiety, a hinge domain (e.g., CD8 hinger such as SEQ ID NO: 53), a transmembrane domain (e.g., a CD8 transmembrane domain such as SEQ ID NO: 38), a co-stimulatory domain (e.g., a 4-1BB co-stimulatory domain such as SEQ ID NO: 39), an IL2Rb signaling domain (e.g., SEQ ID NO: 40), and a cytoplasmic signaling domain (e.g., a CD3z signaling domain such as SEQ ID NO: 42, or 43). In some instances, the bi-specific CAR polypeptide may further comprise a signal peptide at the N-terminus, for example, the exemplary signal peptides provided in Sequence Table 1 (SEQ ID NOs: 45-52)

In some examples, the bi-specific CAR polypeptide is specific to CD19 and BCMA and comprises the above noted components. Examples include SEQ ID NOs: 64, 66, 68, or 70 (mature polypeptide) and SEQ ID NOs: 63, 65, 67, or 69 (include the N-terminus signal peptide).

### II. Genetically Engineered Immune Cells Expressing Bi-Specific CAR

In one aspect, the present disclosure provides a population of immune cells (e.g., T cells) comprising genetically engineered immune cells (e.g., T cells) that express any of the bi-specific CAR polypeptides described herein. The population of immune cells may further comprise one or more disrupted endogenous proinflammatory cytokine genes. As used herein, the term “endogenous” refers to naturally originating from within an organism. Alternatively or in addition, the genetically engineered immune cells that express any of the bi-specific CAR polypeptides may further express one or more antagonists (e.g., exogenous) targeting the proinflammatory cytokines. Such genetically engineered immune cells would have inhibited signaling mediated by the proinflammatory cytokine in in vivo. In some instances, the genetically engineered immune cells disclosed herein may exhibit inhibition of more than one cytokine signaling in vivo.

For purpose of the present disclosure, it will be explicitly understood that the term “antagonist” encompass all the identified terms, titles, and functional states and characteristics whereby the target protein itself, a biological activity of the target protein, or the consequences of the biological activity, are substantially nullified, decreased, or neutralized in any meaningful degree, e.g., by at least 20%, 50%, 70%, 85%, 90%, or above.

Non-limitation examples of proinflammatory cytokines include IL2, IL1α, IL1β, IL-5, IL-6, IL-7, IL-8, IL-9, IL-12, IL-15, IL-17, IL-18, IL-21, IL-23, sIL-1RI, sIL-2Rα, sIL6R, IFNα, IFNβ, IFNγ, MIPα, MIPβ, CSF1, LIF, G-CSF, GM-CSF, CXCL10, CCL5, eotaxin, TNF, MCP1, MIG, RAGE, CRP, angiopoietin-2, VWF, TGFα, VEGF, EGF, HGF, FGF, perforin, granzyme, and ferritin. In some instances, the proinflammatory cytokines includes interferon gamma (IFNγ), interleukin 6 (IL-6), granulocyte-macrophage colony-stimulating factor (GM-CSF), interleukin 1 (IL-1), or a combination thereof.

### A. Immune Cells

Any immune cells may be used to engineer the cells described herein. In some embodiments, an immune cell can be derived, for example without limitation, from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. In other embodiments, the immune cell is derived from the differentiation of a population of induced pluripotent cells (iPSCs).

Useful immune cells for making the engineer the cells disclosed herein may be T-cells, NK cells, tumor infiltrating lymphocytes, dendritic cells, macrophages, B cells, neutrophils, eosinophils, basophils, mast cells, myeloid-derived suppressor cells, mesenchymal stem cells, precursors thereof, or combinations thereof. The T-cells may be selected from the group consisting of inflammatory T-lymphocytes, cytotoxic T-lymphocytes, regulatory T-lymphocytes or helper T-lymphocytes. In some embodiments, the T-cells can be derived from the group consisting of CD4+T-lymphocytes and CD8+T-lymphocytes. In one example, the immune cell is a human immune cell. Representative human immune cells are CD34+ cells.

In some embodiments, the immune cells may be harvested directly from a subject, e.g., a human subject. The cells are genetically modified as described herein and the genetically engineered immune cells are infused back into the same subject, for example, in a CAR-T cell therapy. In this case, the genetically engineered immune cells are autologous to the subject receiving the CAR-T cell therapy. In another embodiment, the immune cells may be harvested directly from a donor subject, modified, and the genetically engineered immune cells are infused into a recipient subject in need of therapy, e.g., a CAR-T cell therapy. The donor immune cells are HLA-matched with to the recipient subject, i.e., the cells are allogeneic to the recipient subject. In some embodiments, the immune cells are harvested from the peripheral blood of the subject, expanded in vitro prior to genetically modification as disclosed herein.

### B. Antagonists of Proinflammatory Cytokines

In some instances, the genetically engineered immune cells disclosed herein may be engineered to express one or more antagonists against proinflammatory cytokines, e.g., those disclosed herein. In some examples, the antagonists are IL-6 antagonistic antibodies, e.g., anti-IL6 antibodies, anti-IL6R antibodies, or anti-gp130 antibodies. Alternatively or in addition, the genetically engineered immune cells may be engineered to express one or more IL-1 antagonists, e.g., IL-1RA or others known in the art or disclosed herein. Alternatively or in addition, the genetically engineered immune cells may be engineered to express one or more IFNγ antagonists, e.g., an antagonistic IFNγ antibody or others known in the art or disclosed herein.

A typical antibody molecule as disclosed herein comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also the Human Genome Mapping Project Resources at the Medical Research Council in the United Kingdom and the antibody rules described at the Bioinformatics and Computational Biology group website at University College London.

An antibody (interchangeably used in plural form) as used herein is an immunoglobulin molecule capable of specific binding to a target protein, e.g., IL-6 or IL-6R, through at least one antigen recognition site, located in the variable region of the immunoglobulin molecule. As used herein, the term “antibody” encompasses not only intact (e.g., full-length) antibodies and heavy chain antibodies (e.g., an Alpaca heavy chain IgG antibody), but also antigen-binding fragments thereof (such as Fab, Fab′, F(ab′)2, Fv), single chain (scFv), single-domain antibody (sdAb; VHH), also known as a nanobody, mutants thereof, fusion proteins comprising an antibody portion, humanized antibodies, chimeric antibodies, diabodies, linear antibodies, single chain antibodies, multispecific antibodies (e.g., bi-specific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, the antibodies described herein that “bind” a target protein or a receptor thereof may specifically bind to the target protein or receptor. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target cytokine if it binds with greater affinity, avidity, more readily, and/or with greater duration than it binds to other substances. For example, an antibody that specifically (or preferentially) binds to an IL-6 or an IL-6R epitope is an antibody that binds this IL-6 epitope or IL-6R epitope with greater affinity, avidity, more readily, and/or with greater duration than it binds to other IL-6 epitopes, non-IL-6 epitopes, other IL-6R epitopes or non-IL-6R epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

The antibodies described herein can be murine, rat, human, or any other origin (including chimeric or humanized antibodies). Such antibodies are non-naturally occurring, e.g., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, and/or six), which are altered with respect to the original antibody, which are also termed one or more CDRs “derived from” one or more CDRs from the original antibody. Humanized antibodies may also involve affinity maturation.

In some embodiments, an antagonistic antibody of a target protein as described herein has a suitable binding affinity for the target protein (e.g., human IL-6, human IL-6R, or human IFNγ) or antigenic epitopes thereof. As used herein, “binding affinity” refers to the apparent association constant or KA. The KA is the reciprocal of the dissociation constant (KD). The antagonistic antibody described herein may have a binding affinity (KD) of at least 10-5, 106, 10-7, 10-8, 10-9, 10-10 M, or lower for the target antigen or antigenic epitope. An increased binding affinity corresponds to a decreased KD. Higher affinity binding of an antibody for a first antigen relative to a second antigen can be indicated by a higher KA (or a smaller numerical value KD) for binding the first antigen than the KA (or numerical value KD) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the antagonistic antibodies described herein have a higher binding affinity (a higher KA or smaller KD) to the target protein in mature form as compared to the binding affinity to the target protein in precursor form or another protein, e.g., an inflammatory protein in the same family as the target protein. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 105 fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Some examples are provided below.

### (a) Antagonistic Antibodies Targeting IL6 Signaling

In some embodiments, the genetically engineered immune cells expressing the bi-specific CAR polypeptide described herein may also express an IL-6 antagonist.

IL-6 signals through a complex comprising the membrane glycoprotein gp130 and the IL-6 receptor (IL-6R) (see, e.g., Hibi et al., Cell, 63(6):1149-57, 1990). IL-6 binding to IL-6R on target cells promotes gp130 homo-dimerization and subsequent signal transduction. As used herein, IL-6R includes both membrane bound and soluble forms of IL-6R (sIL-6R). When bound to IL-6, soluble IL-6R (sIL-6R) acts as an agonist and can also promote gp130 dimerization and signaling. Trans-signaling can occur whereby sIL-6R secretion by a particular cell type induces cells that only express gp130 to respond to IL-6 (see, e.g., Taga et al., Annu Rev Immunol., 15:797-819, 1997; and Rose-John et al., Biochem J., 300 (Pt 2):281-90, 1994). In one example, sIL-6R comprises the extracellular domain of human IL-6R (see e.g., Peters et al., J Exp Med., 183(4): 1399-406, 1996).

In some embodiments, the modified immune cells disclosed herein express an IL-6 antagonist, which may be an antibody that binds to IL-6 or to an IL-6 receptor (IL-6R, including gp130). Such antibodies (antagonistic antibodies) can interfere with binding of IL-6/IL-6R on immune cells, thereby suppressing cell signaling mediated by IL-6.

In some embodiments, the IL-6 antagonistic antibody as described herein can bind and inhibit the IL-6 signaling by at least 50% (e.g., 60%, 70%, 80%, 90%, 95% or greater). The inhibitory activity of an IL-6 antagonistic antibody described herein can be determined by routine methods known in the art.

The heavy chain variable domains (V_{H}) and light chain variable domains (V_{L}) of exemplary anti-IL-6 antibodies and anti-IL-6R antibodies are provided below (Reference Antibodies 1-6) with the CDRs shown in boldface (determined following the antibody rules described by the Bioinformatics and Computational Biology group website at University College London).

Exemplary antibodies that inhibit the IL-6 signaling pathway, including anti-IL-6 antibodies, anti-IL-6R antibodies, and anti-gp130 antibodies, are provided in Sequence Table 1 (AB1-AB6, and IL6 antagonist scFv1-scFv4), all of which are within the scope of the present disclosure.

In some embodiments, the IL-6 antagonistic antibodies described herein bind to the same epitope in an IL-6 antigen (e.g., human IL-6) or in an IL-6R (e.g., human IL-6R) as one of the reference antibodies provided herein (e.g., any one of AB1-AB6 such as AB1 or AB2) or compete against the reference antibody from binding to the IL-6 or IL-6R antigen. Reference antibodies provided herein include Antibodies 1-6, the structural features and binding activity of each of which are provided herein. An antibody that binds the same epitope as a reference antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the reference antibody. Whether two antibodies compete against each other from binding to the cognate antigen can be determined by a competition assay, which is well known in the art. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the IL-6 or IL-6R antigen.

In some instances, the IL-6 antagonistic antibodies disclosed herein may comprise the same heavy chain CDRs and/or the same light chain CDRs as a reference antibody as disclosed herein (e.g., e.g., any one of AB1-AB6 such as AB1 or AB2). The heavy chain and/or light chain CDRs are the regions/residues that are responsible for antigen binding; such regions/residues can be identified from amino acid sequences of the heavy chain/light chain sequences of the reference antibody (shown above) by methods known in the art. See, e.g., antibody rules described at the Bioinformatics and Computational Biology group website at University College London; Almagro, J. Mol. Recognit. 17:132-143 (2004); Chothia et al., J. Mol. Biol. 227:799-817 (1987), as well as others known in the art or disclosed herein. Determination of CDR regions in an antibody is well within the skill of the art, for example, the methods disclosed herein, e.g., the Kabat method (Kabat et al. Sequences of Proteins of Immunological Interest, (5th ed., 1991, National Institutes of Health, Bethesda Md.)) or the Chothia method (Chothia et al., 1989, Nature 342:877; Al-lazikani et al (1997) J. Molec. Biol. 273:927-948)). As used herein, a CDR may refer to the CDR defined by any method known in the art. Two antibodies having the same CDR means that the two antibodies have the same amino acid sequence of that CDR as determined by the same method.

Also within the scope of the present disclosure are functional variants of any of the exemplary anti-IL-6 or anti-IL-6R antibodies as disclosed herein (e.g., any one of AB1-AB6, such as AB1 or AB2). A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, or a combination thereof) as the reference antibody.

In some examples, the IL-6 antagonistic antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as any one of AB1-AB6, e.g., AB1 or AB2. “Collectively” means that the total number of amino acid variations in all of the three HC CDRs is within the defined range. Alternatively or in addition, the anti-IL-6 or anti-IL-6R antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody.

In some examples, the IL-6 antagonistic antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody (e.g., any one of AB1-AB6 such as AB1 or AB2). In specific examples, the antibody comprises a HC CDR3, which contains no more than 5 amino acid variations (e.g., no more than 4, 3, 2, or 1 amino acid variation) as the HC CDR3 of a reference antibody (e.g., any one of AB1-AB6 such as AB1 or AB2). Alternatively or in addition, an IL-6 antagonistic antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody.

In some instances, the amino acid residue variations can be conservative amino acid residue substitutions. See disclosures herein.

In some embodiments, the IL-6 antagonistic antibody disclosed herein may comprise heavy chain CDRs that collectively are at least 80% (e.g., 85%, 90%, 95%, or 98%) identical to the heavy chain CDRs of a reference antibody such as any one of AB1-AB6, e.g., AB1 or AB2. Alternatively or in addition, the antibody may comprise light chain CDRs that collectively are at least 80% (e.g., 85%, 90%, 95%, or 98%) identical to the light chain CDRs of the reference antibody. In some embodiments, the IL-6 antagonistic antibody may comprise a heavy chain variable region that is at least 80% (e.g., 85%, 90%, 95%, or 98%) identical to the heavy chain variable region of a reference antibody such as any one of AB1-AB6, e.g., AB1 or AB2; and/or a light chain variable region that is at least 80% (e.g., 85%, 90%, 95%, or 98%) identical to the light chain variable region of the reference antibody.

The present disclosure also provides germlined variants of any of the reference IL-6 antagonistic antibodies disclosed herein. A germlined variant contains one or more mutations in the framework regions as relative to its parent antibody towards the corresponding germline sequence. To make a germlined variant, the heavy or light chain variable region sequence of the parent antibody or a portion thereof (e.g., a framework sequence) can be used as a query against an antibody germline sequence database (e.g., the antibody rules described at the Bioinformatics and Computational Biology group website at University College London; thevbase2 website, or the IMGT®, the international ImMunoGeneTics information system® website) to identify the corresponding germline sequence used by the parent antibody and amino acid residue variations in one or more of the framework regions between the germline sequence and the parent antibody. One or more amino acid substitutions can then be introduced into the parent antibody based on the germline sequence to produce a germlined variant.

In some examples, the antagonistic antibodies described herein are human antibodies or humanized antibodies. Alternatively or in addition, the antagonistic antibodies are scFv. Exemplary scFv antibodies are provided in Sequence Table 2 below.

### (b) IL-1 Antagonists

In some embodiments, the genetically engineered immune cells expressing the bi-specific CAR described herein may also express an IL-1 antagonist.

Interleukin-1 is a cytokine known in the art and includes two isoforms, IL-1α and IL-1β. IL-1 plays important roles in up- and down-regulation of acute inflammation, as well as other biological pathways.

In some examples, the IL-1 antagonist expressed in the genetically engineered immune cells disclosed herein can be an interleukin-1 receptor antagonist (IL-1RA). IL-1RA is a naturally-occurring polypeptide, which can be secreted by various types of cells, such as immune cells, epithelial cells, and adipocytes. It binds to cell surface IL-1R receptor and thereby preventing the cell signaling triggered by IL-1/IL-1R interaction. A human IL-1RA is encoded by the IL1RN gene. In one example, a human IL-1RA comprising the amino acid sequence of SEQ ID NO: 54 (a mature protein). In some instances, the human-IL-1RA may comprise a signal peptide at the N-terminus, e.g., comprising the amino acid sequence of SEQ ID NO: 55 or SEQ ID NO: 56.

Other IL-1 antagonists include, but are not limited to, anti-IL-1α or anti-IL-1β antibodies (see Fredericks Z L, et al., 2004, Protein Eng Des Sel. 17(1):95-106); U.S. Pat. Nos. 7,531,166 and 8,383,778, the contents are incorporated herein by reference in their entireties.

### (c) IFNγ Antagonists

In some embodiments, the genetically engineered immune cell described herein may express an IFNγ antagonist, in combination with the bi-specific CAR disclosed herein, optionally also in combination with one or more additional genetic modifications as also disclosed herein.

The IFNγ antagonist may block the formation of the ternary IFNγ/IFNγR1/IFNγR2. IFNγ R1 is required for ligand binding and signaling. The IFNγ antagonist can be an antagonistic anti-IFNγ antibody or antigen-binding fragment thereof; a secreted IFNγ receptor or a ligand-binding fragment of the receptor; and an antagonistic anti-IFNγR antibody or antigen-binding fragment thereof, whereby the IFNγ antagonist blocks IFNγ/IFNγR interaction and downstream signaling. In one embodiment, the IFNγ antagonist is secreted. The antagonistic anti-IFNγ antibody or antigen-binding fragment thereof binds the IFNγ ligand that is released in vivo and thus the IFNγ ligand is not available to interact with its native receptor, IFNγR1, expressed on cell surfaces. The secreted IFNγ receptor or a ligand-binding fragment functions as decoy receptor and captures the IFNγ ligand that is released in vivo and thus the IFNγ ligand is also not available to interact with its native receptor, IFNγR1 that is expressed on cell surfaces. In one embodiment, the secreted IFNγR or a ligand-binding fragment is the extracellular portion of a native human IFNγ receptor. The antagonistic anti-IFNγR antibody or antigen-binding fragment thereof binds to the IFNγ receptor expressed on cells and prevents the interaction of the IFNγ ligand with the receptor and the consequential ligand-induced assembly of the complete receptor complex that contains two IFNγR1 and two IFNγR2 subunits. The complete receptor complex is necessary for the IFNγ signaling pathway.

In some embodiments, the modified immune cells disclosed herein express an IFNγ antagonistic antibody. In some examples, the IFNγ antagonistic antibody as described herein can inhibit the IFNγ signaling by at least 50% (e.g., 60%, 70%, 80%, 90%, 95% or greater). The inhibitory activity of an IFNγ antagonistic antibody described herein can be determined by routine methods known in the art.

The heavy chain variable domains (V_{H}) and light chain variable domains (V_{L}) of exemplary anti-IFNγ antibodies and anti-IL-6R antibodies are provided in Sequence Table 1 below (Reference Anti-IFNγ 1-3) with the CDRs in boldface and underlined (based on the Kabat definition).

In some embodiments, the IFNγ antagonistic antibodies described herein bind to the same epitope in an IFNγ antigen (e.g., human IFNγ) as one of the reference antibodies provided herein (e.g., any one of Anti-IFNγ 1-3) or compete against the reference antibody from binding to the IFNγ antigen. Reference antibodies provided herein include Anti-IFNγ 1-3, the structural features and binding activity of each of which are provided herein. See Sequence Table 2. In one example, the anti-human IFN-γ antibody may be derived from AMG811, are described in U.S. Pat. No. 7,335,743, the relevant portions of which are incorporated herein by reference for the subject matter and purpose referenced herein. Alternatively, the anti-human IFN-γ antibody may be derived from fontolizumab or emapalumab. Other antagonistic anti-IFNγ antibodies or antigen-binding fragments thereof can be found in U.S. Pat. No. 9,682,142, the content of which is incorporated by reference for the subject matter and purpose referenced herein.

In some instances, the IFNγ antagonistic antibodies disclosed herein may comprise the same heavy chain CDRs and/or the same light chain CDRs as a reference antibody as disclosed herein (e.g., e.g., any one of Anti-IFNγ 1-3).

Also within the scope of the present disclosure are functional variants of any of the exemplary anti-IFNγ antibodies as disclosed herein (e.g., any one of Anti-IFNγ 1-3). A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, or a combination thereof) as the reference antibody.

In some examples, the IFNγ antagonistic antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as any one of Anti-IFNγ1-3. Alternatively or in addition, the anti-IFNγ antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody.

In some examples, the IFNγ antagonistic antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody (e.g., any one of Anti-IFNγ 1-3). In specific examples, the antibody comprises a HC CDR3, which contains no more than 5 amino acid variations (e.g., no more than 4, 3, 2, or 1 amino acid variation) as the HC CDR3 of a reference antibody (e.g., any one of Anti-IFNγ 1-3). Alternatively or in addition, an IFNγ antagonistic antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody.

In some instances, the amino acid residue variations can be conservative amino acid residue substitutions. See disclosures herein.

In some embodiments, the IFNγ antagonistic antibody disclosed herein may comprise heavy chain CDRs that collectively are at least 80% (e.g., 85%, 90%, 95%, or 98%) identical to the heavy chain CDRs of a reference antibody such as any one of Anti-IFNγ 1-3. Alternatively or in addition, the antibody may comprise light chain CDRs that collectively are at least 80% (e.g., 85%, 90%, 95%, or 98%) identical to the light chain CDRs of the reference antibody. In some embodiments, the IFNγ antagonistic antibody may comprise a heavy chain variable region that is at least 80% (e.g., 85%, 90%, 95%, or 98%) identical to the heavy chain variable region of a reference antibody such as any one of Anti-IFNγ 1-3 and/or a light chain variable region that is at least 80% (e.g., 85%, 90%, 95%, or 98%) identical to the light chain variable region of the reference antibody.

The present disclosure also provides germlined variants of any of the reference IFNγ antagonistic antibodies disclosed herein. In some examples, the antagonistic antibodies described herein are human antibodies or humanized antibodies. Alternatively or in addition, the antagonistic antibodies are scFv. Exemplary scFv antibodies are provided in Sequence Table 2 below.

In other embodiments, the INFγ antagonists disclosed herein may be soluble IFNγR fragments, for example, the extracellular portion of a native human IFNγ receptor. Exemplary IFNγR fragments are known in the art, for example, described in U.S. Pat. Nos. 5,578,707 and 7,449,176, the relevant disclosures of each of which are incorporated by reference for the subject matter and purpose referenced herein. The high-affinity IFNγ receptor complex is made up of two type I membrane proteins, IFNγR1 (IFNγR alpha) and IFNγR2 (IFNγR beta). Both proteins are members of the type II cytokine receptor family and share approximately 52% overall sequence identity. IFNγR1 is the ligand-binding subunit that is necessary and sufficient for IFNγ binding and receptor internalization. IFNγR2 is required for IFNγ signaling but does not bind IFNγ by itself. Human IFNγR1 cDNA encodes a 499 amino acid (aa) residue protein with a 17 aa signal peptide, a 228 aa extracellular domain, a 23 aa transmembrane domain, and a 221 aa intracellular domain. Soluble IFNγR fragments that antagonizes the IFNγ signaling may comprises the 228 aa extracellular domain.

In yet other embodiments, the IFNγ antagonists disclosed herein can be antagonistic anti-IFNγR antibodies or antigen-binding fragments thereof, for example, those described in U.S. Pat. Nos. 4,897,264 and 7,449,176, the relevant disclosures of which are incorporated by reference for the subject matter and purpose referenced herein.

Any of the IFNγ antagonists described herein may comprising a signal peptide located at the N-terminus of the IFNγ antagonist so that it can be secreted by the genetically engineered immune cells expressing such. Exemplary signal peptides are provided in the Sequence Table 2, any of which can be used in the IFNγ antagonist.

### C. Disruption of Endogenous Proinflammatory Cytokine Genes

In some embodiments, the genetically engineered immune cells expressing any of the bi-specific CARs disclosed herein, optionally also expressing one or more of the antagonists also disclosed herein, may have one or more disrupted endogenous proinflammatory cytokine genes (e.g., the GM-CSF gene and/or the IFNγ gene). Some examples are provided below.

### (a) Disruption of Endogenous Interferon Gamma Gene

In some instances, the genetically engineered immune cells disclosed herein are genetically engineered to provide a reduced level of IFNγ as compared with counterpart immune cells without such a genetic modification, e.g., at least 20%, at least 30%, at least 40%, at least 50%, at least 60%, at least 70%, at least 80%, at least 90%, or at least 95% lower compared to the counterpart immune cells. The amount of IFNγ produced by such genetically engineered immune cells may be determined by any method know in the art, e.g., by an ELISA assay of the cell culture media or the blood IFNγ level of a patient treated with such modified cells.

In other instances, the genetically engineered immune cells may reduce a reduced level of IFNγR (e.g., IFNγR1) as compared with the counterpart immune cells that do not have such a genetic modification, e.g., at least 20%, at least 30%, at least 40%, at least 50%, at least 60%, at least 70%, at least 80%, at least 90%, or at least 95% lower compared to the counterpart immune cells.

In some examples, reduction of IFNγ may be achieved by disrupting an endogenous IFNγ gene and/or an endogenous IFNγR gene, e.g., by genetic editing. Such genetically engineered immune cells, which express any of the bi-specific CARs disclosed herein, would be expected to have limited cytokine release syndrome mediated by the IFNγ signaling in vivo.

Any methods known in the art for down-regulating the expression of an endogenous gene in a host cell, including gene editing, can be used to reduce the expression level of IFNγ or IFNγR as described herein. The genomic information for the human IFNγ and IFNγR1 are found in GENBANK Gene ID: 3458 and Gene ID: 3459 respectively.

In some examples, a gene editing method may be used to disrupt an endogenous IFNγ or IFNγR gene. The gene editing system may involve an endonuclease that is capable of cleaving a target region in the endogenous allele. Non-homologous end joining in the absence of a template nucleic acid may repair double-strand breaks in the genome and introduce mutations (e.g., insertions, deletions and/or frameshifts) into a target site.

In some examples, a knocking-out event can be coupled with a knocking-in event—an exogenous nucleic acid coding for a desired molecule (e.g., the IL6 antagonist, the IFNγ antagonist or the IL1 antagonist described herein) can be inserted into a genomic locus of the IFNγ or IFNγR gene via gene editing in combination with homologous recombination, to insert the exogenous nucleic acid at the target genomic site, thereby disrupting the endogenous target gene.

In one example, disrupting an endogenous IFNγ or IFNγR gene can be achieved via a CRISPR/Cas-mediated gene editing method, for example, using a CRISPR/Cas9-mediated gene editing system. To disrupt the IFNγ gene, a guide RNA (gRNA) specific to a target site adjuvant to a protospacer adjacent motif (PAM) may be used. The sgRNAs molecules contains both the custom-designed short crRNA sequence fused to the scaffold tracrRNA sequence. Exemplary genetic target sites in the human IFNγ gene (e.g., in exon 1), the corresponding spacer sequences of gRNAs, and exemplary single guide RNAs (sgRNA) are provided in Sequence Table 3. Any of these gRNAs can be used to disrupt the human IFNγ gene.

To disrupt the IFNγR gene, commercially available IFNγR1 Human Gene Knockout Kit (CRISPR) Cat #KN202761 from OriGene Technologies may be used. Methods of using such kits are known in the art.

In other instances, reduction of the level of IFNγ or IFNγR can be achieved by antisense oligonucleotides via the antisense technology or by interfering RNAs (e.g., shRNAs or siRNAs) via the RNA interference technology. Alternatively, ribozymes may be used to achieve this goal. An antisense oligonucleotide or interfering RNA is an oligonucleotide that comprises a fragment complementary to a target region of an endogenous target gene or a transcript thereof. Such antisense oligonucleotides can be delivered into target cells via conventional methods. Alternatively, expression vectors such as lentiviral vectors or equivalent thereof can be used to express such an antisense oligonucleotide or interfering RNA.

### D. Populations of Genetically Engineered Immune Cells

In some aspects, provided herein is a population of genetically engineered immune cells expressing any of the bi-specific CARs described herein (e.g., an anti-CD19/anti-BCMA bi-specific CAR), and comprising one or more additional genetic modifications, e.g., engineered to express one or more antagonists targeting proinflammatory cytokines, engineered to reduce the expression of endogenous proinflammatory cytokines (e.g., via disruption of the endogenous gene by, e.g., gene editing), or a combination thereof.

In some examples, the genetically engineered immune cells expressing a bi-specific CAR as disclosed herein (e.g., an anti-CD19/anti-BCMA bi-specific CAR) may further express an antagonistic antibody (e.g., an scFv antibody) inhibiting the IL6 signaling, an antagonistic antibody (e.g., an scFv antibody) inhibiting the IFNγ signaling, an IL1 antagonist, or a combination thereof. Examples of such antagonistic agents are disclosed herein.

Alternatively or in addition, the genetically engineered immune cells disclosed herein may contain one or more disrupted endogenous genes encoding one or more proinflammatory cytokines (e.g., IFNγ or GM-CSF). The genetically engineered immune cells may comprise further genetic editing in genes of interest, for example, the gene encoding a TCR component or the gene encoding a MHC Class I or MHC Class II component. In some instances, a nucleic acid encoding any of the antagonistic agent disclosed herein may be inserted at the disrupted gene locus.

The population of genetically engineered immune cells may be heterogenous, comprising cells having different genetic modifications or different combination of genetic modifications. For example, a subgroup of cells in the population may co-express the bi-specific CAR and an antagonist of a proinflammatory cytokine and another subgroup of cells in the population may express the bi-specific CAR and have a disrupted endogenous target gene. The cells in the population, collectively, have all of the desired genetic modifications as disclosed herein. In some instances, a portion of the immune cell population may exhibit all of the desired genetic modifications in each cell, e.g., (a) expressing the bi-specific CAR, in combination with expressing an IL6 antagonist and/or an IFNγ antagonist, (b) expressing the bi-specific CAR, in combination with knocking down an endogenous IFNγ gene and/or GM-CSF gene, or (c) expressing the bi-specific CAR, in combination with expressing an IL6 antagonist and/or an IFNγ antagonist and knocking down an endogenous IFNγ gene and/or GM-CSF gene. In some examples, such a portion may constitute at least 20% (e.g., at least 30%, at least 40%, or at least 50%) of the total population of genetically engineered immune cells as disclosed herein.

Specific knock-in and knock-out genetic modifications for CAR-T cells, including the IFNγ antagonists, IL-6 antagonists and IL-1 antagonists, can be found in WO2019/178259 and WO2020/146239, the relevant disclosures of each of which are incorporated by reference for the purpose and subject matter disclosed herein.

### III. Methods of Preparing Genetically Engineered Immune Cells

Any of the knock-in and knock-out modifications may be introduced into suitable immune cells by routine methods and/or approaches described herein. Typically, such methods would involve delivery of genetic material into the suitable immune cells to either down-regulate expression of a target endogenous inflammatory protein, express a cytokine antagonist of interest or express an immune suppressive cytokine of interest.

### (A) Knocking in Modification

To generate a knock-in of one or more bi-specific CARs, IFNγ antagonists, IL-6 antagonists, and IL-1 antagonists described herein, a coding sequence of the one or more the bi-specific CARs, IFNγ antagonists, IL-6 antagonists, and IL-1 antagonists may be cloned into a suitable expression vector (e.g., including but not limited to lentiviral vectors, retroviral vectors, adenoviral vectors, adeno-associated vectors, PiggyBac transposon vector and Sleeping Beauty transposon vector) and introduced into host immune cells using conventional recombinant technology. Sambrook et al., Molecular Cloning, A Laboratory Manual, 3rd Ed., Cold Spring Harbor Laboratory Press. As a result, modified immune cells of the present disclosure may comprise one or more exogenous nucleic acids encoding at least one bi-specific CAR, IFNγ antagonists, IL-6 antagonist, or IL-1 antagonist. In some instances, the coding sequence of such molecules is integrated into the genome of the cell. In some instances, the coding sequence of such molecules is not integrated into the genome of the cell.

Knock-in refers to introduce an exogenous nucleic acid into host cells. In some instances, the exogenous nucleic acid may be inserted into a genomic site of the host cells (e.g., for stable expression of the encoded gene product). Alternatively, the exogenous nucleic acid may exist extrachromosomal (e.g., for transient expression of the encoded gene product).

An exogenous nucleic acid comprising a coding sequence of interest may further comprise a suitable promoter, which can be in operable linkage to the coding sequence. A promoter, as used herein, refers to a nucleotide sequence (site) on a nucleic acid to which RNA polymerase can bind to initiate the transcription of the coding DNA (e.g., for a cytokine antagonist) into mRNA, which will then be translated into the corresponding protein (i.e., expression of a gene). A promoter is considered to be “operably linked” to a coding sequence when it is in a correct functional location and orientation relative to the coding sequence to control (“drive”) transcriptional initiation and expression of that coding sequence (to produce the corresponding protein molecules). In some instances, the promoter described herein can be constitutive, which initiates transcription independent other regulatory factors. In some instances, the promoter described herein can be inducible, which is dependent on regulatory factors for transcription. Exemplary promoters include, but are not limited to ubiquitin, RSV, CMV, EF1α and PGK1. In one example, one or more nucleic acids encoding one or more antagonists of one or more inflammatory cytokines as those described herein, operably linked to one or more suitable promoters can be introduced into immune cells via conventional methods to drive expression of one or more antagonists.

Additionally, the exogenous nucleic acids described herein may further contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Suitable methods for producing vectors containing transgenes are well known and available in the art. Sambrook et al., Molecular Cloning, A Laboratory Manual, 3rd Ed., Cold Spring Harbor Laboratory Press.

In some instances, one or more bi-specific CARs, IFNγ antagonists, IL-6 antagonists, or IL-1 antagonists can be constructed in one expression cassette in a multi-cistronic manner such that the various molecules are expressed as separate polypeptides. In some examples, an internal ribosome entry site can be inserted between two coding sequences to achieve this goal. Alternatively, a nucleotide sequence coding for a self-cleaving peptide (e.g., T2A or P2A) can be inserted between two coding sequences. Exemplary designs of such multi-cistronic expression cassettes are provided in Examples below.

### (B) Knocking Out Modification

Any methods known in the art for down-regulating the expression of an endogenous gene in a host cell can be used to reduce the production level of a target endogenous cytokine/protein as described herein. A gene editing method may involve use of an endonuclease that is capable of cleaving the target region in the endogenous allele. Non-homologous end joining in the absence of a template nucleic acid may repair double-strand breaks in the genome and introduce mutations (e.g., insertions, deletions and/or frameshifts) into a target site. Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), endonucleases (e.g., ARC homing endonucleases), meganucleases (e.g., mega-TALs), or a combination thereof.

Various gene editing systems using meganucleases, including modified meganucleases, have been described in the art; see, e.g., the reviews by Steentoft et al., Glycobiology 24(8): 663-80, 2014; Belfort and Bonocora, Methods Mol Biol. 1123:1-26, 2014; Hafez and Hausner, Genome 55(8):553-69, 2012; and references cited therein. In some examples, a knocking-out event can be coupled with a knocking-in event—an exogenous nucleic acid coding for a desired molecule such as those described herein can be inserted into a locus of a target endogenous gene of interest via gene editing.

Alternatively, any of the knock-out modification may be achieved using antisense oligonucleotides (e.g., interfering RNAs such as shRNA or siRNA) or ribozymes via methods known in the art. An antisense oligonucleotide specific to a target cytokine/protein refers to an oligonucleotide that is complementary or partially complementary to a target region of an endogenous gene of the cytokine or an mRNA encoding such. Such antisense oligonucleotides can be delivered into target cells via conventional methods. Alternatively, expression vectors such as lentiviral vectors or equivalent thereof can be used to express such an antisense oligonucleotides.

### (C) Preparation of Immune Cell Population Comprising Modified Immune Cells

A population of immune cells comprising any of the modified immune cells described herein, or a combination thereof, may be prepared by introducing into a population of host immune cells one or more of the knock-in modifications, one or more of the knock-out modifications, or a combination thereof. The knock-in and knock-out modifications can be introduced into the host cells in any order.

In some instances, one or more modifications are introduced into the host cells in a sequential manner without isolation and/or enrichment of modified cells after a preceding modification event and prior to the next modification event. In that case, the resultant immune cell population may be heterogeneous, comprising cells harboring different modifications or different combination of modifications. Such an immune cell population may also comprise unmodified immune cells. The level of each modification event occurring in the immune cell population can be controlled by the amount of genetic materials that induce such modification as relative to the total number of the host immune cells. See also above discussions.

In other instances, modified immune cells may be isolated and enriched after a first modification event before performing a second modification event. This approach would result in the production of a substantially homogenous immune cell population harboring all of the knock-in and/or knock-out modifications introduced into the cells.

In some examples, the knock-in modification(s) and the knock-out modification(s) are introduced into host immune cells separately. For example, a knock-out modification is performed via gene editing to knock out an endogenous gene for a target cytokine and a knock-in modification is performed by delivering into the host immune cells a separate exogenous expression cassette for producing one or more cytokine antagonists. In some instances, the knock-in and knock-out event can be occurred simultaneously, for example, the knock-in cassette can be inserted into the locus of a target gene to be knocked-out.

### IV. Therapeutic Applications

In some aspects, this disclosure provides a cell therapy-based method of treating a disease or disorder, comprising administering to a subject in need thereof the population of immune cells described herein or a pharmaceutical composition described herein. Any of the immune cell populations comprising the modified immune cells as described herein may be used in an adoptive immune cell therapy (i.e., CAR-T) for treating a target disease, such as leukemia or lymphoma. Due to the knock-in and knock-out modifications introduced into the immune cells, particularly the knock-in of the CAR, the knock-in of the IL-6 antagonistic antibody, the IL-1 antagonist, or a combination thereof, the therapeutic uses of such would be expected to improve proliferation of the therapeutic cells, while achieving the same or better therapeutic effects.

To practice the therapeutic methods described herein, an effective amount of the immune cell population, comprising any of the modified immune cells as described herein, may be administered to a subject who needs treatment via a suitable route (e.g., intravenous infusion). One or more of the immune cell populations may be mixed with a pharmaceutically acceptable carrier to form a pharmaceutical composition prior to administration, which is also within the scope of the present disclosure. The immune cells may be autologous to the subject, i.e., the immune cells are obtained from the subject in need of the treatment, modified to reduce expression of one or more target cytokines/proteins, for example, those described herein, to express one or more cytokine antagonists described herein, to express a CAR construct and/or exogenous TCR, or a combination thereof. The resultant modified immune cells can then be administered to the same subject. Administration of autologous cells to a subject may result in reduced rejection of the immune cells as compared to administration of non-autologous cells. Alternatively, the immune cells can be allogeneic cells, i.e., the cells are obtained from a first subject, modified as described herein and administered to a second subject that is different from the first subject but of the same species. For example, allogeneic immune cells may be derived from a human donor and administered to a human recipient who is different from the donor.

In one embodiment, prior to the cell therapy, the subject received a lymphodepleting treatment to condition the subject for the cell therapy. Examples of lymphodepleting treatment comprises administering to the subject one or more of fludarabine and cyclophosphamide.

The subject to be treated may be a mammal (e.g., human, mouse, pig, cow, rat, dog, guinea pig, rabbit, hamster, cat, goat, sheep or monkey). The subject may be suffering from cancer, have an infectious disease or an immune disorder. Exemplary cancers include but are not limited to hematologic malignancies (e.g., B-cell acute lymphoblastic leukemia, chronic lymphocytic leukemia and multiple myeloma). Exemplary infectious diseases include but are not to human immunodeficiency virus (HIV) infection, Epstein-Barr virus (EBV) infection, human papillomavirus (HPV) infection, dengue virus infection, malaria, sepsis and Escherichia coli infection. Exemplary immune disorders include but are not limited to, autoimmune diseases, such as rheumatoid arthritis, type I diabetes, systemic lupus erythematosus, inflammatory bowel disease, multiple sclerosis, Guillain-Barre syndrome, chronic inflammatory demyelinating polyneuropathy, psoriasis, Graves' disease, Hashimoto's thyroiditis, myasthenia gravis, and vasculitis.

In some instances, the genetically engineered immune cells such as T cells disclosed herein express a bi-specific CAR targeting both CD19 and BCMA (e.g., those disclosed herein). Such bi-specific CAR-T cells can be used to treat human patients having a CD19+ and/or BCMA+ cancer (e.g., a hematological cancer or a solid tumor). In some examples, the cancer may be lymphoblastic leukemia, acute lymphoblastic leukemia, chronic lymphoblastic leukemia, mantle cell lymphoma, large B-cell lymphoma, or non-Hodgkin's lymphoma. In other examples, the cancer may be multiple myeloma, relapsed multiple myeloma, or refractory multiple myeloma. Alternatively, the human patient may have breast cancer, gastric cancer, neuroblastoma, or osteosarcoma.

In some embodiments, the CAR-T cells described herein are useful for treating B-cell related cancers. Non-limiting B-cell related cancers include multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, follicular lymphoma, marginal zone lymphoma, mucosa-associated lymphatic tissue lymphoma, small cell lymphocytic lymphoma, mantle cell lymphoma, Burkitt lymphoma, primary mediastinal (thymic) large B-cell lymphoma, lymphoplasmactyic lymphoma, Waldenström macroglobulinemia, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBV positive diffuse large B-cell lymphoma of the elderly, diffuse large B-cell lymphoma associated with inflammation, intravascular large B-cell lymphoma, ALK-positive large B-cell lymphoma, plasmablastic lymphoma (PBL), large B-cell lymphoma arising in HHV8-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other B-cell related lymphoma.

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

An effective amount of the immune cells may be administered to a human patient in need of the treatment via a suitable route, e.g., intravenous infusion. In some instances, about 1×10⁶ to about 1×10⁸ CAR+ T cells may be given to a human patient (e.g., a leukemia patient, a lymphoma patient, or a multiple myeloma patient). In some examples, a human patient may receive multiple doses of the immune cells. For example, the patient may receive two doses of the immune cells on two consecutive days. In some instances, the first dose is the same as the second dose. In other instances, the first dose is lower than the second dose, or vice versa.

In any of the treatment methods disclosed herein, which involves the use of the immune cells, the subject may be administered IL-2 concurrently with the cell therapy. More specifically, an effective amount of IL-2 may be given to the subject via a suitable route before, during, or after the cell therapy. In some embodiments, IL-2 is given to the subject after administration of the immune cells.

Alternatively or in addition, the subject being treated by the cell therapy disclosed herein may be free from treatment involving an IL-6 antagonist (aside from an IL-6 antagonist produced by the immune cells used in the cell therapy) after immune cell infusion.

The immune cell populations comprising the modified immune cells as described herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

In some embodiments, the method of treating cancer does not elicit severe CRS in the subject being treated within 14 days of infusion of the genetically engineered cells. In one embodiment of the treatment methods, the subject being treated may not need to receive additional anti-IL-6 therapy such as tocilizumab. In some embodiments, the subject being treated may not need to receive steroid therapy to suppress the immune system. In other embodiments, the subject being treated may receive immunosuppressive steroids such as methylprednisolone and dexamethasone in conjunction with infusion of the immune cells disclosed herein. A skilled clinician will be able to determine the vital signs and symptoms of the subject to monitor and assess for the grade/severity of CRS during treatment and timely administer appropriate medication to suppress the developing CRS.

In some examples, the subject is subject to a suitable anti-cancer therapy (e.g., those disclosed herein) to reduce tumor burden prior to the CAR-T therapy disclosed herein. For example, the subject (e.g., a human cancer patient) may be subject to a chemotherapy (e.g., comprising a single chemotherapeutic agent or a combination of two or more chemotherapeutic agents) at a dose that substantially reduces tumor burden. In some instances, the chemotherapy may reduce the total white blood cell count in the subject to lower than 10⁸/L, e.g., lower than 10⁷/L. Tumor burden of a patient after the initial anti-cancer therapy, and/or after the CAR-T cell therapy disclosed herein may be monitored via routine methods. If a patient showed a high growth rate of cancer cells after the initial anti-cancer therapy and/or after the CAR-T therapy, the patient may be subject to a new round of chemotherapy to reduce tumor burden followed by any of the CAR-T therapy as disclosed herein.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, interferon gamma, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation or radiation and chemotherapy is used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of

### Diagnosis and Therapy, (1992), Merck Research Laboratories, Rahway N.J.

### V. Kits for Therapeutic Uses or Making Genetically Engineered Immune Cells

The present disclosure also provides kits for use of any of the target diseases described herein involving one or more of the immune cell population described herein and kits for use in making the modified immune cells as described herein.

A kit for therapeutic use as described herein may include one or more containers comprising an immune cell population, which may be formulated to form a pharmaceutical composition. The immune cell population comprises any of the modified immune cells described herein or a combination thereof. The population of immune cells, such as T lymphocytes, NK cells, and others described herein may further express a bi-specific CAR construct as described herein.

In some embodiments, the kit can additionally comprise instructions for use of the immune cell population in any of the methods described herein. The included instructions may comprise a description of administration of the immune cell population or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the immune cell population or the pharmaceutical composition comprising such to a subject who is in need of the treatment.

The instructions relating to the use of the immune cell population or the pharmaceutical composition comprising such as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of immune cells (e.g., T lymphocytes or NK cells) that comprise any of the modified immune cells or a combination thereof.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Also provided here are kits for use in making the modified immune cells as described herein. Such a kit may include one or more containers each containing reagents for use in introducing the knock-in and/or knock-out modifications into immune cells. For example, the kit may contain one or more components of a gene editing system for making one or more knock-out modifications as those described herein. Alternatively or in addition, the kit may comprise one or more exogenous nucleic acids for expressing cytokine antagonists as also described herein and reagents for delivering the exogenous nucleic acids into host immune cells. Such a kit may further include instructions for making the desired modifications to host immune cells.

### General Techniques

The practice of the present disclosure will employ, unless otherwise indicated, conventional techniques of molecular biology (including recombinant techniques), microbiology, cell biology, biochemistry, immunology and chimeric antigen receptor (CAR) immunotherapy, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane, Cold Spring Harbor Laboratory Press, 1999); The Antibodies (M. Zanetti and J. D. Capra, eds., Harwood Academic Publishers, 1995); DNA Cloning: A practical Approach, Volumes I and II (D. N. Glover ed. 1985); Nucleic Acid Hybridization (B. D. Hames & S. J. Higgins eds. 1985); Transcription and Translation (B. D. Hames and S. J. Higgins, eds. 1984); Animal Cell Culture (R. I. Freshney, ed., 1986); Immobilized Cells and Enzymes ((B. Perbal, IRL Press, 1986); A practical Guide To Molecular Cloning (F. M. Ausubel et al., eds 1984); Chimeric Antigen Receptor (CAR) Immunotherapy (D. W. Lee and N. N. Shah, eds., Elservier, 2019, ISBN: 9780323661812); Basics of Chimeric Antigen Receptor (CAR) Immunotherapy (M. Y. Balkbi, Academic Press, Elsevier Science, 2019, ISBN: 9780128197479); Chimeric Antigen Receptor T Cells Development and Production (V. Picanço-Castro, K. C. R. Malmegrim, K. Swiech, eds., Springer US, 2020, ISBN: 9781071601488); Cell and Gene Therapies (C. Bollard, S. A. Abutalib, M.-A. Perales eds., Springer International, 2018; ISBN: 9783319543680) and Developing Costimulatory Molecules for Immunotherapy of Diseases (M. A. Mir, Elsevier Science, 2015, ISBN: 9780128026755).

The present disclosure is not limited in its application to the details of construction and the arrangements of component set forth in the description herein or illustrated in the drawings. The present disclosure is capable of other embodiments and of being practice or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. As also used in this specification and the appended claims, the singular forms “a,” “an,” and “the” include plural references unless the context clearly dictates otherwise.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

SEQUENCE TABLE 1
Antibody Sequences.

| | | Sequence | |
|---|---|---|---|
| | | (CDRs in boldface and underlined; | SEQ |
| | | determined by the Kabat | ID |
| Description | | method) | NO |
| Anti-CD19 VHH 1 | | EVQLLESGGGLVQPGGSLRSCEASGFNAMTWVRQPPGKG | 1 |
| | | LEWVSSIDSWTDAVKGRFAISQDNAKNTVYLQMNSLKPE | |
| | | DTAMYYCALSKCYTRVYDYWGQGTQVTVSS | |
| Anti-CD19 VHH 2 | | EVQLQESGGGLVQPGGSLRLSCAASGFIYMVWVRQAPGK | 2 |
| | | GLEWLSGIKTERDGVKGRFTIPRDNAKNTLYLQMNNLKS | |
| | | EDTALYYCATEENDWGQGTQVTVSS | |
| Anti-CD19 VHH 3 | | QVKLEESGGELVQPGGPLRLSCAASGNIFSINRMGWYRQA | 3 |
| | | PGKQRAFVASITVRGITNYADSVKGRFTISVDKSKNTIYL | |
| | | QMNALKPEDTAVYYCNAVSSNRDPDYWGQGTQVTVSS | |
| Anti-BCMA VHH 1 | | EVQLLESGGGLVQPGGSLRLSCAASGFTFSSYAMSWVRQA | 4 |
| | | PGKGLEWVSSISGSGDYIYYADSVKGRFTISRDISKNTLY | |
| | | LQMNSLRAEDTAVYYCAKEGTGANSSLADYRGQGTLVTVS | |
| | | S | |
| Anti-BCMA VHH 2 | | QVQLVESGGGLVQPGGSLRLSCAASGFTFSSHAMTWVRQ | 5 |
| | | APGKGLEWVAAISGSGDFTHYADSVKGRFTISRDNSKNTV | |
| | | SLQMNNLRAEDTAVYYCAKDEDGGSLLGYRGQGTLVTV | |
| | | SS | |
| Anti-BCMA VHH 3 | | EVQLLESGGGLIQPGGSLRLSCAASGFTFSSHAMTWVRQA | 6 |
| | | PGKGLEWVSAISGSGDYTHYADSVKGRFTISRDNSKNTVY | |
| | | LQMNSLRAEDSAVYYCAKDEDGGSLLGHRGQGTLVTVSS | |
| Anti-CD19 scFv 1 | | DIQMTQTTSSLSASLGDRVTISCRASQDISKYLNWYQQK | 7 |
| | | PDGTVKLLIYHTSRLHSGVPSRFSGSGSGTDYSLTISNL | |
| | | EQEDIATYFCQQGNTLPYTFGGGTKLEITGSTSGSGKPG | |
| | | SGEGSTKGEVKLQESGPGLVAPSQSLSVTCTVSGVSLPD | |
| | | YGVSWIRQPPRKGLEWLGVIWGSETTYYNSALKSRLTII | |
| | | KDNSKSQVFLKMNSLQTDDTAIYYCAKHYYYGGSYAMDY | |
| | | WGQGTSVTVSS | |
| Anti-CD19 scFv 2 | | DVVMTQSPSSIPVTLGESVSISCRSSKSLQNVNGNTYLYWF | 8 |
| | | QQRPGQSPQLLIYRMSNLNSGVPDRFSGSGSGTDFTLRISG | |
| | | VEPEDVGVYYCMQHLEYPLTFGAGTKLEIKGGGGSGGG | |
| | | GSGGGGSQVQLVQSGPELIKPGGSVKMSCKASGYTFTSYV | |
| | | MHWVRQKPGQGLEWIGYINPYNDGTKYNEKFKGRATLT | |
| | | SDKSSSTAYMELSSLRSEDSAVYYCARGTYYYGSRVFDY | |
| | | WGQGTTVTVSS | |
| Anti-CD19 scFv 3 | | DVVMTQSPSSIPVTLGESVSISCRSSKSLQNVNGNTYLYWF | 9 |
| | | QQRPGQSPQLLIYRMSNLNSGVPDRESGSGSGTDFTLRISG | |
| | | VEPEDVGVYYCMQHLEYPLTFGAGTKLEIKGGGGSGGG | |
| | | GSGGGGSQVQLVQSGPELIKPGGSVKMSCKASGYTFTSYV | |
| | | MHWVRQKPGQGLEWIGYINPYNDGTKYNEKFKGRATLT | |
| | | SDKSSSTAYMELSSLRSEDSAVYYCARGTYYYGSRVFDY | |
| | | WGQGTTVTVSS | |
| Anti-BCMA scFv | | DIVLTQSPPSLAMSLGKRATISCRASESVTILGSHLIHWYQ | 10 |
| | | QKPGQPPTLLIQLASNVQTGVPARFSGSGSRTDFTLTIDPVE | |
| | | EDDVAVYYCLQSRTIPRTFGGGTKLEIKGSTSGSGKPGSG | |
| | | EGSTKGQIQLVQSGPELKKPGETVKISCKASGYTFTDYSIN | |
| | | WVKRAPGKGLKWMGWINTETREPAYAYDFRGRFAFSLE | |
| | | TSASTAYLQINNLKYEDTATYFCALDYSYAMDYWGQGTS | |
| | | VTVSS | |
| Anti-BCMA | | EVQLLESGGGLIQPGGSLRLSCAASGFTFSSHAMTWVRQAP | 11 |
| VHH/anti-CD19 | | GKGLEWVSAISGSGDYTHYADSVKGRFTISRDNSKNTVYL | |
| scFv | | QMNSLRAEDSAVYYCAKDEDGGSLLGHRGQGTLVTVSSG | |
| | | GGGSPAGDIQMTQTTSSLSASLGDRVTISCRASQDISKYLNWY | |
| | | QQKPDGTVKLLIYHTSRLHSGVPSRFSGSGSGTDYSLTISNLEQ | |
| | | EDIATYFCQQGNTLPYTFGGGTKLEITGSTSGSGKPGSGEGST | |
| | | KGEVKLQESGPGLVAPSQSLSVTCTVSGVSLPDYGVSWIRQPP | |
| | | RKGLEWLGVIWGSETTYYNSALKSRLTIIKDNSKSQVFLKMNSL | |
| | | QTDDTAIYYCAKHYYYGGSYAMDYWGQGTSVTVSS | |
| Anti-CD19 VHH/ | | QVKLEESGGELVQPGGPLRLSCAASGNIFSINRMGWYRQA | 12 |
| anti-BCMA scFv | | PGKQRAFVASITVRGITNYADSVKGRFTISVDKSKNTIYLQ | |
| | | MNALKPEDTAVYYCNAVSSNRDPDYWGQGTQVTVSSGG | |
| | | GGSPAGDIVLTQSPPSLAMSLGKRATISCRASESVTILGSHL | |
| | | IHWYQQKPGQPPTLLIQLASNVQTGVPARFSGSGSRTDFTL | |
| | | TIDPVEEDDVAVYYCLQSRTIPRTFGGGTKLEIKGSTSGSGK | |
| | | PGSGEGSTKGQIQLVQSGPELKKPGETVKISCKASGYTFTDYSI | |
| | | NWVKRAPGKGLKWMGWINTETREPAYAYDFRGRFAFSLETSA | |
| | | STAYLQINNLKYEDTATYFCALDYSYAMDYWGQGTSVTVSS | |
| Anti CD19 scFv/ | | DIQMTQTTSSLSASLGDRVTISCRASQDISKYLNWYQQKPDGTV | 71 |
| anti-BCMA VHH | | KLLIYHTSRLHSGVPSRFSGSGSGTDYSLTISNLEQEDIATYFCQ | |
| | | QGNTLPYTFGGGTKLEITGSTSGSGKPGSGEGSTKGEVKL | |
| | | QESGPGLVAPSQSLSVTCTVSGVSLPDYGVSWIRQPPRKGLEW | |
| | | LGVIWGSETTYYNSALKSRLTIIKDNSKSQVFLKMNSLQTDDTAI | |
| | | YYCAKHYYYGGSYAMDYWGQGTSVTVSSGGGGSPAGEVQLL | |
| | | ESGGGLIQPGGSLRLSCAASGFTFSSHAMTWVRQAPGKGL | |
| | | EWVSAISGSGDYTHYADSVKGRFTISRDNSKNTVYLQMNS | |
| | | LRAEDSAVYYCAKDEDGGSLLGHRGQGTLVTVSS | |
| Anti BCMA scFv/ | | DIVLTQSPPSLAMSLGKRATISCRASESVTILGSHLIHWYQQ | 72 |
| anti-CD19 VHH | | KPGQPPTLLIQLASNVQTGVPARFSGSGSRTDFTLTIDPVEE | |
| | | DDVAVYYCLQSRTIPRTFGGGTKLEIKGSTSGSGKPGSGE | |
| | | GSTKGQIQLVQSGPELKKPGETVKISCKASGYTFTDYSINW | |
| | | VKRAPGKGLKWMGWINTETREPAYAYDFRGRFAFSLETS | |
| | | ASTAYLQINNLKYEDTATYFCALDYSYAMDYWGQGTSVT | |
| | | VSSGGGGSPAGQVKLEESGGELVQPGGPLRLSCAASGNIF | |
| | | SINRMGWYRQAPGKQRAFVASITVRGITNYADSVKGRFTIS | |
| | | VDKSKNTIYLQMNALKPEDTAVYYCNAVSSNRDPDYWGQ | |
| | | GTQVTVSS | |
| Anti- | VH | QVQLVQSGAELKKPGSSVKVSCKASGYIFTSSWINWVKQA | 13 |
| IFNγ1 | | PGQGLEWIGRIDPSDGEVHYNQDFKDKATLTVDKSTNTA | |
| | | YMELSSLRSEDTAVYYCARGFLPWFADWGQGTLVTVSS | |
| | VL | DIQMTQSPSTLSASVGDRVTITCKASENVDTYVSWYQQKP | 14 |
| | | GKAPKLLIYGASNRYTGVPSRFSGSGSGTDFTLTISSLQPDD | |
| | | FATYYCGQSYNYPFTFGQGTKVEVKR | |
| Anti-IFNγ | | DIQMTQSPSTLSASVGDRVTITCKASENVDTYVSWYQQKP | 15 |
| scFv1 | | GKAPKLLIYGASNRYTGVPSRFSGSGSGTDFTLTISSLQPDD | |
| | | FATYYCGQSYNYPFTFGQGTKVEVKRGGGGSGGGGSGG | |
| | | GGSQVQLVQSGAELKKPGSSVKVSCKASGYIFTSSWINWV | |
| | | KQAPGQGLEWIGRIDPSDGEVHYNQDFKDKATLTVDKST | |
| | | NTAYMELSSLRSEDTAVYYCARGFLPWFADWGQGTLVT | |
| | | VSS | |
| Anti- | VH | EVQLLESGGGLVQPGGSLRLSCAASGFTFSSYAMSWVRQA | 16 |
| IFNγ2 | | PGKGLEWVSAISGSGGSTYYADSVKGRFTISRDNSKNTLY | |
| | | LQMNSLRAEDTAVYYCAKDGSSGWYVPHWFDPWGQGT | |
| | | LVTVSS | |
| | VL | NFMLTQPHSVSESPGKTVTISCTRSSGSIASNYVQWYQQRP | 17 |
| | | GSSPTTVIYEDNQRPSGVPDRESGSIDSSSNSASLTISGLKTE | |
| | | DEADYYCQSYDGSNRWMFGGGTKLTVL | |
| Anti-IFNγ | | NFMLTQPHSVSESPGKTVTISCTRSSGSIASNYVQWYQQRP | 18 |
| scFv2 | | GSSPTTVIYEDNQRPSGVPDRESGSIDSSSNSASLTISGLKTE | |
| | | DEADYYCQSYDGSNRWMFGGGTKLTVLGGGGSGGGGS | |
| | | GGGGSEVQLLESGGGLVQPGGSLRLSCAASGFTESSYAMS | |
| | | WVRQAPGKGLEWVSAISGSGGSTYYADSVKGRFTISRDN | |
| | | SKNTLYLQMNSLRAEDTAVYYCAKDGSSGWYVPHWFDP | |
| | | WGQGTLVTVSS | |
| Anti- | VH | EVQLVQSGAEVKKPGESLKISCKGSGYNFTSYWIGWVRQ | 19 |
| IFNγ3 | | MPGKGLELMGIIYPGDSDTRYSPSFQGQVTISADKSISTAY | |
| | | LQWSSLKASDTAMYYCGSGSYFYFDLWGRGTLVTVSS | |
| | VL | EIVLTQSPGTLSLSPGERATLSCRASQSVSSSYLAWYQQKP | 20 |
| | | GQAPRLLIYGASSRATGIPDRESGSGSGTDFTLTISRLEPEDF | |
| | | AVYYCQRSGGSSFTFGPGTKVDIK | |
| Anti-IFNγ | | EIVLTQSPGTLSLSPGERATLSCRASQSVSSSYLAWYQQKP | 21 |
| scFv 3 | | GQAPRLLIYGASSRATGIPDRFSGSGSGTDFTLTISRLEPEDF | |
| | | AVYYCQRSGGSSFTFGPGTKVDIKGGGGSGGGGSGGGG | |
| | | SEVQLVQSGAEVKKPGESLKISCKGSGYNFTSYWIGWVRQ | |
| | | MPGKGLELMGIIYPGDSDTRYSPSFQGQVTISADKSISTAY | |
| | | LQWSSLKASDTAMYYCGSGSYFYFDLWGRGTLVTVSS | |
| AB1 | VH | EVQLVESGGGLVQPGRSLRLSCAASRFTFDDYAMHWVRQ | 22 |
| (anti- | | APGKGLEWVSGISWNSGRIGYADSVKGRFTISRDNAENSL | |
| IL6R) | | FLQMNGLRAEDTALYYCAKGRDSFDIWGQGTMVTVSS | |
| | VL | DIQMTQSPSSVSASVGDRVTITCRASQGISSWLAWYQQKP | 23 |
| | | GKAPKLLIYGASSLESGVPSRFSGSGSGTDFTLTISSLQPEDF | |
| | | ASYYCQQANSFPYTFGQGTKLEIK | |
| AB2 | VH | EVQLVESGGGLVQPGGSLRLSCAASGFTFSPFAMSWVRQA | 24 |
| (anti- | | PGKGLEWVAKISPGGSWTYYSDTVTGRFTISRDNAKNSL | |
| IL6) | | YLQMNSLRAEDTAVYYCARQLWGYYALDIWGQGTTVTV | |
| | | SS | |
| | VL | EIVLTQSPATLSLSPGERATLSCSASISVSYMYWYQQKPGQ | 25 |
| | | APRLLIYDMSNLASGIPARFSGSGSGTDFTLTISSLEPEDFAV | |
| | | YYCMQWSGYPYTFGGGTKVEIK | |
| AB3 | VH | EVQLVESGGKLLKPGGSLKLSCAASGFTFSSFAMSWFRQSP | 26 |
| (anti- | | EKRLEWVAEISSGGSYTYYPDTVTGRFTISRDNAKNTLYL | |
| IL6) | | EMSSLRSEDTAMYYCARGLWGYYALDYWGQGTSVTVSS | |
| | VL | QIVLIQSPAIMSASPGEKVTMTCSASSSVSYMYWYQQKPGS | 27 |
| | | SPRLLIYDTSNLASGVPVRESGSGSGTSYSLTISRMEAEDAA | |
| | | TYYCQQWSGYPYTFGGGTKLEIK | |
| AB4 | VH | QVQLQESGPGLVRPSQTLSLTCTVSGYSITSDHAWSWVRQ | 28 |
| (anti- | | PPGRGLEWIGYISYSGITTYNPSLKSRVTMLRDTSKNQFSL | |
| IL6R) | | RLSSVTAADTAVYYCARSLARTTAMDYWGQGSLVTVSS | |
| | VL | DIQMTQSPSSLSASVGDRVTITCRASQDISSYLNWYQQKPG | 29 |
| | | KAPKLLIYYTSRLHSGVPSRFSGSGSGTDFTFTISSLQPEDIA | |
| | | TYYCQQGNTLPYTFGQGTKVEIK | |
| AB5 | VH | EVQLVESGGGLVQPGGSLRLSCAASGFSLSNYYVTWVRQA | 30 |
| (anti- | | PGKGLEWVGIIYGSDETAYATSAIGRFTISRDNSKNTLYLQ | |
| IL6) | | MNSLRAEDTAVYYCARDDSSDWDAKFNLWGQGTLVTVS | |
| | | S | |
| | VL | AIQMTQSPSSLSASVGDRVTITCQASQSINNELSWYQQKPG | 31 |
| | | KAPKLLIYRASTLASGVPSRFSGSGSGTDFTLTISSLQPDDF | |
| | | ATYYCQQGYSLRNIDNAFGGGTKVEIK | |
| AB6 | VH | EVQLVESGGGLVQPGGSLRLSCAASGFNFNDYFMNWVRQ | 32 |
| (anti- | | APGKGLEWVAQMRNKNYQYGTYYAESLEGRFTISRDDS | |
| gp130) | | KNSLYLQMNSLKTEDTAVYYCARESYYGFTSYWGQGTLV | |
| | | TV | |
| | VL | DIQMTQSPSSLSASVGDRVTITCQASQDIGISLSWYQQKPG | 33 |
| | | KAPKLLIYNANNLADGVPSRFSGSGSGTDFTLTISSLQPEDF | |
| | | ATYYCLQHNSAPYTFGQGTKLEIK | |

| | | |
|---|---|---|
| IL6 antagonist | DIQMTQSPSSVSASVGDRVTITCRASQGISSWLAWYQQKP | 34 |
| scFv 1 | GKAPKLLIYGASSLESGVPSRFSGSGSGTDFTLTISSLQPEDF | |
| | ASYYCQQANSFPYTFGQGTKLEIKGGGGSGGGGSGGGG | |
| | SEVQLVESGGGLVQPGRSLRLSCAASRFTFDDYAMHWVR | |
| | QAPGKGLEWVSGISWNSGRIGYADSVKGRFTISRDNAENS | |
| | LFLQMNGLRAEDTALYYCAKGRDSFDIWGQGTMVTVSS | |
| IL6 antagonist | EIVLTQSPATLSLSPGERATLSCSASISVSYMYWYQQKPGQ | 35 |
| scFv 2 | APRLLIYDMSNLASGIPARFSGSGSGTDFTLTISSLEPEDFAV | |
| | YYCMQWSGYPYTFGGGTKVEIKGGGGSGGGGSGGGGS | |
| | EVQLVESGGGLVQPGGSLRLSCAASGFTFSPFAMSWVRQA | |
| | PGKGLEWVAKISPGGSWTYYSDTVTGRFTISRDNAKNSL | |
| | YLQMNSLRAEDTAVYYCARQLWGYYALDIWGQGTTVTV | |
| | SS | |
| IL6 antagonist | QIVLIQSPAIMSASPGEKVTMTCSASSSVSYMYWYQQKPGS | 36 |
| scFv 3 | SPRLLIYDTSNLASGVPVRESGSGSGTSYSLTISRMEAEDAA | |
| | TYYCQQWSGYPYTFGGGTKLEIKGGGGSGGGGSGGGGS | |
| | EVQLVESGGKLLKPGGSLKLSCAASGFTFSSFAMSWFRQSP | |
| | EKRLEWVAEISSGGSYTYYPDTVTGRFTISRDNAKNTLYL | |
| | EMSSLRSEDTAMYYCARGLWGYYALDYWGQGTSVTVSS | |
| IL6 antagonist | QVQLQESGPGLVRPSQTLSLTCTVSGYSITSDHAWSWVRQ | 37 |
| scFv 4 | PPGRGLEWIGYISYSGITTYNPSLKSRVTMLRDTSKNQFSL | |
| | RLSSVTAADTAVYYCARSLARTTAMDYWGQGSLVTVSSG | |
| | GGGSGGRASGGGGSGGGGSDIQMTQSPSSLSASVGDRVT | |
| | ITCRASQDISSYLNWYQQKPGKAPKLLIYYTSRLHSGVPSR | |
| | FSGSGSGTDFTFTISSLQPEDIATYYCQQGNTLPYTFGQGT | |
| | KVEIK | |

SEQUENCE TABLE 2
Sequences of Chimeric Antigen Receptor and Components Thereof

| | | SEQ ID |
|---|---|---|
| Description | Sequence | NO |
| CD8 | IYIWAPLAGTCGVLLLSLVITLYC | 38 |
| transmembrane | | |
| 4-1BB | KRGRKKLLYIFKQPFMRPVQTTQEEDGCSCRFPEEEEGGCEL | 39 |
| Costimulatory | | |
| domain | | |
| IL-2Rb signaling | NCRNTGPWLKKVLKCNTPDPSKFFSQLSSEHGGDVQKWLSS | 40 |
| domain | PFPSSSFSPGGLAPEISPLEVLERDKVTQLLPLNTDAYLSLQEL | |
| | QGQDPTHLV | |
| STAT binding | YRHQ | 41 |
| motif | | |
| CD3z signaling | RVKFSRSADAPAYKQGQNQLYNELNLGRREEYDVLDKRRG | 42 |
| domain with | RDPEMGGKPRRKNPQEGLYNELQKDKMAEAYSEIGMKGER | |
| STAT binding | RRGKGHDGLYQGLSTATKDTYDAYRHQALPPR | |
| motif | | |
| CD3z signaling | RVKFSRSADAPAYKQGQNQLYNELNLGRREEYDVLDKRRG | 43 |
| domain 1 | RDPEMGGKPRRKNPQEGLYNELQKDKMAEAYSEIGMKGER | |
| | RRGKGHDGLYQGLSTATKDTYDALHMQALPPR | |
| CD8 signal | MALPVTALLLPLALLLHAARP | 45 |
| peptide | | |
| Antibody signal | MKYLLPTAAAGLLLLAAQPAMA | 46 |
| peptide | | |
| Gaussia | MGVKVLFALICIAVAEA | 47 |
| luciferase signal | | |
| peptide | | |
| human albumin | MKWVTFISLLFLESSAYS | 48 |
| signal peptide | | |
| modified human | MKWVTFISLLFLFSSSSRA | 49 |
| albumin signal | | |
| peptide | | |
| modified IL2 | MRRMQLLLLIALSLALVTNS | 50 |
| signal peptide | | |
| growth hormone | MATGSRTSLLLAFGLLCLPWLQEGSA | 51 |
| signal peptide | | |
| native IL-IRA | MALETIC | 52 |
| signal peptide | | |
| CD8 hinge | TTTPAPRPPTPAPTIASQPLSLRPEACRPAAGGAVHTRGLDFA | 53 |
| | CD | |
| IL-1RA (mature) | RPSGRKSSKMQAFRIWDVNQKTFYLRNNQLVAGYLQGPNV | 54 |
| | NLEEKIDVVPIEPHALFLGIHGGKMCLSCVKSGDETRLQLEA | |
| | VNITDLSENRKQDKRFAFIRSDSGPTTSFESAACPGWFLCTA | |
| | MEADQPVSLTNMPDEGVMVTKFYFQEDE | |
| IL-1RA 2 | MATGSRTSLLLAFGLLCLPWLQEGSARPSGRKSSKMQAFRI | 55 |
| | WDVNQKTFYLRNNQLVAGYLQGPNVNLEEKIDVVPIEPHAL | |
| | FLGIHGGKMCLSCVKSGDETRLQLEAVNITDLSENRKQDKRF | |
| | AFIRSDSGPTTSFESAACPGWFLCTAMEADQPVSLTNMPDEG | |
| | VMVTKFYFQEDE | |
| IL-1RA 1 | MALETICRPSGRKSSKMQAFRIWDVNQKTFYLRNNQLVAGY | 56 |
| | LQGPNVNLEEKIDVVPIEPHALFLGIHGGKMCLSCVKSGDET | |
| | RLQLEAVNITDLSENRKQDKRFAFIRSDSGPTTSFESAACPGW | |
| | FLCTAMEADQPVSLTNMPDEGVMVTKFYFQEDE | |
| GS Linker | GGGGSPAG | 57 |
| Peptide Linker | SGGGSDPGGGGSGGGGSGGGGSGGGGS | 73 |
| Peptide Linker | EAAAK | 74 |
| Motif | | |
| G4S linker | GGGGS | 75 |
| (G4S)₃ | GGGGSGGGGSGGGGS | 76 |
| (G4S)₄ | GGGGSGGGGSGGGGSGGGGS | 77 |
| trAPRIL | HSVLHLVPINATSKDDSDVTEVMWQPALRRGRGLQAQGYG | 58 |
| | VRIQDAGVYLLYSQVLFQDVTFTMGQVVSREGQGRQETLFR | |
| | CIRSMPSHPDRAYNSCYSAGVFHLHQGDILSVIIPRARAKLNL | |
| | SPHGTFLGFVKL | |
| trAPRIL/anti- | HSVLHLVPINATSKDDSDVTEVMWQPALRRGRGLQAQGYG | 59 |
| CD19 scFv 1 | VRIQDAGVYLLYSQVLFQDVTFTMGQVVSREGQGRQETLFR | |
| | CIRSMPSHPDRAYNSCYSAGVFHLHQGDILSVIIPRARAKLNL | |
| | SPHGTFLGFVKLSGGGSDPGGGGSGGGGSGGGGSGGGGSPAGD | |
| | IQMTQTTSSLSASLGDRVTISCRASQDISKYLNWYQQKPDGTVK | |
| | LLIYHTSRLHSGVPSRFSGSGSGTDYSLTISNLEQEDIATYFCQ | |
| | QGNTLPYTFGGGTKLEITGSTSGSGKPGSGEGSTKGEVKLQESG | |
| | PGLVAPSQSLSVTCTVSGVSLPDYGVSWIRQPPRKGLEWLGVIW | |
| | GSETTYYNSALKSRLTIIKDNSKSQVFLKMNSLQTDDTAIYYCA | |
| | KHYYYGGSYAMDYWGQGTSVTVSS | |
| trAPRIL/anti- | HSVLHLVPINATSKDDSDVTEVMWQPALRRGRGLQAQGYG | 60 |
| CD19 scFv 2 | VRIQDAGVYLLYSQVLFQDVTFTMGQVVSREGQGRQETLFR | |
| | CIRSMPSHPDRAYNSCYSAGVFHLHQGDILSVIIPRARAKLNL | |
| | SPHGTFLGFVKLGGGGSPAGDIQMTQTTSSLSASLGDRVTISCR | |
| | ASQDISKYLNWYQQKPDGTVKLLIYHTSRLHSGVPSRFSGSGSGT | |
| | DYSLTISNLEQEDIATYFCQQGNTLPYTFGGGTKLEITGSTSGSGK | |
| | PGSGEGSTKGEVKLQESGPGLVAPSQSLSVTCTVSGVSLPDYGVS | |
| | WIRQPPRKGLEWLGVIWGSETTYYNSALKSRLTIIKDNSKSQVFL | |
| | KMNSLQTDDTAIYYCAKHYYYGGSYAMDYWGQGTSVTVSS | |
| trAPRIL/anti- | HSVLHLVPINATSKDDSDVTEVMWQPALRRGRGLQAQGYG | 61 |
| CD19 VHH 1 | VRIQDAGVYLLYSQVLFQDVTFTMGQVVSREGQGRQETLFR | |
| | CIRSMPSHPDRAYNSCYSAGVFHLHQGDILSVIIPRARAKLNL | |
| | SPHGTFLGFVKLSGGGSDPGGGGSGGGGSGGGGSGGGGSQVK | |
| | LEESGGELVQPGGPLRLSCAASGNIFSINRMGWYRQAPGKQRAF | |
| | VASITVRGITNYADSVKGRFTISVDKSKNTIYLQMNALKPEDT | |
| | AVYYCNAVSSNRDPDYWGQGTQVTVSS | |
| trAPRIL/anti- | HSVLHLVPINATSKDDSDVTEVMWQPALRRGRGLQAQGYG | 62 |
| CD19 VHH | VRIQDAGVYLLYSQVLFQDVTFTMGQVVSREGQGRQETLFR | |
| | CIRSMPSHPDRAYNSCYSAGVFHLHQGDILSVIIPRARAKLNL | |
| | SPHGTFLGFVKLGGGGSPAGQVKLEESGGELVQPGGPLRLSC | |
| | AASGNIFSINRMGWYRQAPGKQRAFVASITVRGITNYADSVKGRF | |
| | TISVDKSKNTIYLQMNALKPEDTAVYYCNAVSSNRDPDYWGQGT | |
| | QVTVSS | |
| Anti CD19 scFv/ | DIQMTQTTSSLSASLGDRVTISCRASQDISKYLNWYQQKPDG | 44 |
| anti-BCMAVHH | TVKLLIYHTSRLHSGVPSRFSGSGSGTDYSLTISNLEQEDIATY | |
| Bispecific CAR | FCQQGNTLPYTFGGGTKLEITGSTSGSGKPGSGEGSTKGEVK | |
| | LQESGPGLVAPSQSLSVTCTVSGVSLPDYGVSWIRQPPRKGL | |
| | EWLGVIWGSETTYYNSALKSRLTIIKDNSKSQVFLKMNSLQT | |
| | DDTAIYYCAKHYYYGGSYAMDYWGQGTSVTVSSGGGGSP | |
| | AGEVQLLESGGGLIQPGGSLRLSCAASGFTFSSHAMTWVRQ | |
| | APGKGLEWVSAISGSGDYTHYADSVKGRFTISRDNSKNTVY | |
| | LQMNSLRAEDSAVYYCAKDEDGGSLLGHRGQGTLVTVSS G | |
| | STTTPAPRPPTPAPTIASQPLSLRPEACRPAAGGAVHTRGLDFAC | |
| | DIYIWAPLAGTCGVLLLSLVITLYCKRGRKKLLYIFKQPFMRPVQT | |
| | TQEEDGCSCRFPEEEEGGCELNCRNTGPWLKKVLKCNTPDPSK | |
| | FFSQLSSEHGGDVQKWLSSPFPSSSFSPGGLAPEISPLEVLERDK | |
| | VTQLLPLNTDAYLSLQELQGQDPTHLVRVKFSRSADAPAYKQGQ | |
| | NQLYNELNLGRREEYDVLDKRRGRDPEMGGKPRRKNPQEGLY | |
| | NELQKDKMAEAYSEIGMKGERRRGKGHDGLYQGLSTATKDTYD | |
| | AYRHQALPPR | |
| Anti CD19 scFv/ | MALPVTALLLPLALLLHAARPDIQMTQTTSSLSASLGDRVTISC | 63 |
| anti-BCMA | RASQDISKYLNWYQQKPDGTVKLLIYHTSRLHSGVPSRFSGS | 64 (no |
| VHH Bispecific | GSGTDYSLTISNLEQEDIATYFCQQGNTLPYTFGGGTKLEITG | signal |
| CAR | STSGSGKPGSGEGSTKGEVKLQESGPGLVAPSQSLSVTCTVS | peptide) |
| | GVSLPDYGVSWIRQPPRKGLEWLGVIWGSETTYYNSALKSR | |
| | LTIIKDNSKSQVFLKMNSLQTDDTAIYYCAKHYYYGGSYAM | |
| | DYWGQGTSVTVSSGGGGSPAGEVQLLESGGGLIQPGGSLRL | |
| | SCAASGFTFSSHAMTWVRQAPGKGLEWVSAISGSGDYTHYA | |
| | DSVKGRFTISRDNSKNTVYLQMNSLRAEDSAVYYCAKDEDG | |
| | GSLLGHRGQGTLVTVSSGSTTTPAPRPPTPAPTIASQPLSLRPEA | |
| | CRPAAGGAVHTRGLDFACDIYIWAPLAGTCGVLLLSLVITLYCKR | |
| | GRKKLLYIFKQPFMRPVQTTQEEDGCSCRFPEEEEGGCELNCR | |
| | NTGPWLKKVLKCNTPDPSKFFSQLSSEHGGDVQKWLSSPFPSSS | |
| | FSPGGLAPEISPLEVLERDKVTQLLPLNTDAYLSLQELQGQDPTH | |
| | LVRVKFSRSADAPAYKQGQNQLYNELNLGRREEYDVLDKRRGR | |
| | DPEMGGKPRRKNPQEGLYNELQKDKMAEAYSEIGMKGERRRG | |
| | KGHDGLYQGLSTATKDTYDAYRHQALPPRSG | |
| Anti CD19 | QVKLEESGGELVQPGGPLRLSCAASGNIFSINRMGWYRQAP | 78 |
| VHH/ anti- | GKQRAFVASITVRGITNYADSVKGRFTISVDKSKNTIYLQMN | |
| BCMA scFv | ALKPEDTAVYYCNAVSSNRDPDYWGQGTQVTVSSGGGGSP | |
| Bispecific CAR | AGDIVLTQSPPSLAMSLGKRATISCRASESVTILGSHLIHWYQ | |
| | QKPGQPPTLLIQLASNVQTGVPARFSGSGSRTDFTLTIDPVEE | |
| | DDVAVYYCLQSRTIPRTFGGGTKLEIKGSTSGSGKPGSGEGS | |
| | TKGQIQLVQSGPELKKPGETVKISCKASGYTFTDYSINWVKR | |
| | APGKGLKWMGWINTETREPAYAYDFRGRFAFSLETSASTAY | |
| | LQINNLKYEDTATYFCALDYSYAMDYWGQGTSVTVSSGSTT | |
| | TPAPRPPTPAPTIASQPLSLRPEACRPAAGGAVHTRGLDFACDIYI | |
| | WAPLAGTCGVLLLSLVITLYCKRGRKKLLYIFKQPFMRPVQTTQE | |
| | EDGCSCRFPEEEEGGCELNCRNTGPWLKKVLKCNTPDPSKFFS | |
| | QLSSEHGGDVQKWLSSPFPSSSFSPGGLAPEISPLEVLERDKVTQ | |
| | LLPLNTDAYLSLQELQGQDPTHLVRVKFSRSADAPAYKQGQNQL | |
| | YNELNLGRREEYDVLDKRRGRDPEMGGKPRRKNPQEGLYNELQ | |
| | KDKMAEAYSEIGMKGERRRGKGHDGLYQGLSTATKDTYDAYRH | |
| | QALPPR | |
| Anti CD19 | MALPVTALLLPLALLLHAARPQVKLEESGGELVQPGGPLRLSC | 65 |
| VHH/ anti- | AASGNIFSINRMGWYRQAPGKQRAFVASITVRGITNYADSVK | 66 (no |
| BCMA scFv | GRFTISVDKSKNTIYLQMNALKPEDTAVYYCNAVSSNRDPD | signal |
| Bispecific CAR | YWGQGTQVTVSSGGGGSPAGDIVLTQSPPSLAMSLGKRATI | peptide) |
| | SCRASESVTILGSHLIHWYQQKPGQPPTLLIQLASNVQTGVPA | |
| | RFSGSGSRTDFTLTIDPVEEDDVAVYYCLQSRTIPRTFGGGTK | |
| | LEIKGSTSGSGKPGSGEGSTKGQIQLVQSGPELKKPGETVKIS | |
| | CKASGYTFTDYSINWVKRAPGKGLKWMGWINTETREPAYA | |
| | YDFRGRFAFSLETSASTAYLQINNLKYEDTATYFCALDYSYA | |
| | MDYWGQGTSVTVSSGSTTTPAPRPPTPAPTIASQPLSLRPEACR | |
| | PAAGGAVHTRGLDFACDIYIWAPLAGTCGVLLLSLVITLYCKRGR | |
| | KKLLYIFKQPFMRPVQTTQEEDGCSCRFPEEEEGGCELNCRNT | |
| | GPWLKKVLKCNTPDPSKFFSQLSSEHGGDVQKWLSSPFPSSSFS | |
| | PGGLAPEISPLEVLERDKVTQLLPLNTDAYLSLQELQGQDPTHLV | |
| | RVKFSRSADAPAYKQGQNQLYNELNLGRREEYDVLDKRRGRDP | |
| | EMGGKPRRKNPQEGLYNELQKDKMAEAYSEIGMKGERRRGKG | |
| | HDGLYQGLSTATKDTYDAYRHQALPPRSG | |
| Anti BCMA | EVQLLESGGGLIQPGGSLRLSCAASGFTFSSHAMTWVRQAPG | 79 |
| VHH/ anti-CD19 | KGLEWVSAISGSGDYTHYADSVKGRFTISRDNSKNTVYLQM | |
| scFv Bispecific | NSLRAEDSAVYYCAKDEDGGSLLGHRGQGTLVTVSSGGGG | |
| CAR | SPAGDIQMTQTTSSLSASLGDRVTISCRASQDISKYLNWYQQ | |
| | KPDGTVKLLIYHTSRLHSGVPSRFSGSGSGTDYSLTISNLEQE | |
| | DIATYFCQQGNTLPYTFGGGTKLEITGSTSGSGKPGSGEGSTK | |
| | GEVKLQESGPGLVAPSQSLSVTCTVSGVSLPDYGVSWIRQPP | |
| | RKGLEWLGVIWGSETTYYNSALKSRLTIIKDNSKSQVFLKMN | |
| | SLQTDDTAIYYCAKHYYYGGSYAMDYWGQGTSVTVSSGST | |
| | TTPAPRPPTPAPTIASQPLSLRPEACRPAAGGAVHTRGLDFACDI | |
| | YIWAPLAGTCGVLLLSLVITLYCKRGRKKLLYIFKQPFMRPVQTT | |
| | QEEDGCSCRFPEEEEGGCELNCRNTGPWLKKVLKCNTPDPSKF | |
| | FSQLSSEHGGDVQKWLSSPFPSSSFSPGGLAPEISPLEVLERDKV | |
| | TQLLPLNTDAYLSLQELQGQDPTHLVRVKFSRSADAPAYKQGQN | |
| | QLYNELNLGRREEYDVLDKRRGRDPEMGGKPRRKNPQEGLYNE | |
| | LQKDKMAEAYSEIGMKGERRRGKGHDGLYQGLSTATKDTYDAY | |
| | RHQALPPR | |
| Anti BCMA | MALPVTALLLPLALLLHAARPEVQLLESGGGLIQPGGSLRLSCA | 67 |
| VHH/ anti-CD19 | ASGFTFSSHAMTWVRQAPGKGLEWVSAISGSGDYTHYADSV | 68 (no |
| scFv Bispecific | KGRFTISRDNSKNTVYLQMNSLRAEDSAVYYCAKDEDGGSL | signal |
| CAR | LGHRGQGTLVTVSSGGGGSPAGDIQMTQTTSSLSASLGDRV | peptide) |
| | TISCRASQDISKYLNWYQQKPDGTVKLLIYHTSRLHSGVPSR | |
| | FSGSGSGTDYSLTISNLEQEDIATYFCQQGNTLPYTFGGGTKL | |
| | EITGSTSGSGKPGSGEGSTKGEVKLQESGPGLVAPSQSLSVTC | |
| | TVSGVSLPDYGVSWIRQPPRKGLEWLGVIWGSETTYYNSAL | |
| | KSRLTIIKDNSKSQVFLKMNSLQTDDTAIYYCAKHYYYGGSY | |
| | AMDYWGQGTSVTVSSGSTTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELNCRN | |
| | TGPWLKKVLKCNTPDPSKFFSQLSSEHGGDVQKWLSSPFPSSSF | |
| | SPGGLAPEISPLEVLERDKVTQLLPLNTDAYLSLQELQGQDPTHL | |
| | VRVKFSRSADAPAYKQGQNQLYNELNLGRREEYDVLDKRRGRD | |
| | PEMGGKPRRKNPQEGLYNELQKDKMAEAYSEIGMKGERRRGK | |
| | GHDGLYQGLSTATKDTYDAYRHQALPPRSG | |
| Anti BCMA | DIVLTQSPPSLAMSLGKRATISCRASESVTILGSHLIHWYQQK | 80 |
| scFv/ anti-CD19 | PGQPPTLLIQLASNVQTGVPARFSGSGSRTDFTLTIDPVEEDD | |
| VHH Bispecific | VAVYYCLQSRTIPRTFGGGTKLEIKGSTSGSGKPGSGEGSTK | |
| CAR | GQIQLVQSGPELKKPGETVKISCKASGYTFTDYSINWVKRAP | |
| | GKGLKWMGWINTETREPAYAYDFRGRFAFSLETSASTAYLQ | |
| | INNLKYEDTATYFCALDYSYAMDYWGQGTSVTVSSGGGGS | |
| | PAGQVKLEESGGELVQPGGPLRLSCAASGNIFSINRMGWYR | |
| | QAPGKQRAFVASITVRGITNYADSVKGRFTISVDKSKNTIYL | |
| | QMNALKPEDTAVYYCNAVSSNRDPDYWGQGTQVTVSSGST | |
| | TTPAPRPPTPAPTIASQPLSLRPEACRPAAGGAVHTRGLDFACDI | |
| | YIWAPLAGTCGVLLLSLVITLYCKRGRKKLLYIFKQPFMRPVQTT | |
| | QEEDGCSCRFPEEEEGGCELNCRNTGPWLKKVLKCNTPDPSKF | |
| | FSQLSSEHGGDVQKWLSSPFPSSSFSPGGLAPEISPLEVLERDKV | |
| | TQLLPLNTDAYLSLQELQGQDPTHLVRVKFSRSADAPAYKQGQN | |
| | QLYNELNLGRREEYDVLDKRRGRDPEMGGKPRRKNPQEGLYNE | |
| | LQKDKMAEAYSEIGMKGERRRGKGHDGLYQGLSTATKDTYDAY | |
| | RHQALPPR | |
| Anti BCMA | MALPVTALLLPLALLLHAARPDIVLTQSPPSLAMSLGKRATISC | 69 |
| scFv/ anti-CD19 | RASESVTILGSHLIHWYQQKPGQPPTLLIQLASNVQTGVPARF | 70 (no |
| VHH Bispecific | SGSGSRTDFTLTIDPVEEDDVAVYYCLQSRTIPRTFGGGTKLE | signal |
| CAR | IKGSTSGSGKPGSGEGSTKGQIQLVQSGPELKKPGETVKISCK | peptide) |
| | ASGYTFTDYSINWVKRAPGKGLKWMGWINTETREPAYAYD | |
| | FRGRFAFSLETSASTAYLQINNLKYEDTATYFCALDYSYAMD | |
| | YWGQGTSVTVSSGGGGSPAGQVKLEESGGELVQPGGPLRL | |
| | SCAASGNIFSINRMGWYRQAPGKQRAFVASITVRGITNYADS | |
| | VKGRFTISVDKSKNTIYLQMNALKPEDTAVYYCNAVSSNRD | |
| | PDYWGQGTQVTVSSGSTTTPAPRPPTPAPTIASQPLSLRPEACR | |
| | PAAGGAVHTRGLDFACDIYIWAPLAGTCGVLLLSLVITLYCKRGR | |
| | KKLLYIFKQPFMRPVQTTQEEDGCSCRFPEEEEGGCELNCRNT | |
| | GPWLKKVLKCNTPDPSKFFSQLSSEHGGDVQKWLSSPFPSSSFS | |
| | PGGLAPEISPLEVLERDKVTQLLPLNTDAYLSLQELQGQDPTHLV | |
| | RVKFSRSADAPAYKQGQNQLYNELNLGRREEYDVLDKRRGRDP | |
| | EMGGKPRRKNPQEGLYNELQKDKMAEAYSEIGMKGERRRGKG | |
| | HDGLYQGLSTATKDTYDAYRHQALPPRSG | |

SEQUENCE TABLE 3
Sequences for Guide RNAs Targeting IFNγ

| | | SEQ ID |
|---|---|---|
| Description | Sequence | NO |
| IFNγ exon 1 | GAAATATACAAGTTATATCT (TGG) | 81 |
| target site 1 | | |
| (PAM) | | |
| sgRNA spacer | GAAAUAUACAAGUUAUAUCU | 82 |
| for target site 1 | | |
| sgRNA for target | GAAAUAUACAAGUUAUAUCUGUUUUAGAGCUAGAAAUA | 83 |
| site 1 | GCAAGUUAAAAUAAGGCUAGUCCGUUAUCAACUUGAAA | |
| | AAGUGGCACCGAGUCGGUGCUUUU | |
| IFNγ exon 1 | TTTCAGCTCTGCATCGTTT (TGG) | 84 |
| target site 2 | | |
| (PAM) | | |
| sgRNA space for | UUUCAGCUCUGCAUCGUUU | 85 |
| target site 2 | | |
| sgRNA for target | GUUUCAGCUCUGCAUCGUUUGUUUUAGAGCUAGAAAUA | 86 |
| site 2 | GCAAGUUAAAAUAAGGCUAGUCCGUUAUCAACUUGAAA | |
| | AAGUGGCACCGAGUCGGUGCUUUU | |
| IFNγ exon 1 | TTCAGCTCTGCATCGTTTT (GGG) | 87 |
| target site 3 | | |
| (PAM) | | |
| sgRNA space for | UUCAGCUCUGCAUCGUUUU | 88 |
| target site 3 | | |
| sgRNA for target | GUUCAGCUCUGCAUCGUUUUGUUUUAGAGCUAGAAAUA | 89 |
| site 3 | GCAAGUUAAAAUAAGGCUAGUCCGUUAUCAACUUGAAA | |
| | AAGUGGCACCGAGUCGGUGCUUUU | |
| IFNγ exon 1 | GCATCGTTTTGGGTTCTCT (TGG) | 90 |
| target site 4 | | |
| (PAM) | | |
| sgRNA space for | GCAUCGUUUUGGGUUCUCU | 91 |
| target site 4 | | |
| sgRNA for target | GCAUCGUUUUGGGUUCUCUGUUUUAGAGCUAGAAAUA | 92 |
| site 4 | GCAAGUUAAAAUAAGGCUAGUCCGUUAUCAACUUGAAA | |
| | AAGUGGCACCGAGUCGGUGCUUUU | |
| IFNγ exon 1 | TCTCTTGGCTGTTACTGCC (AGG) | 93 |
| target site 5 | | |
| (PAM) | | |
| sgRNA space for | UCUCUUGGCUGUUACUGCC | 94 |
| target site 5 | | |
| sgRNA for target | GUCUCUUGGCUGUUACUGCCGUUUUAGAGCUAGAAAU | 95 |
| site 5 | AGCAAGUUAAAAUAAGGCUAGUCCGUUAUCAACUUGAA | |
| | AAAGUGGCACCGAGUCGGUGCUUUU | |
| IFNγ exon 1 | TTCTTTTACATATGGGTCC (TGG) | 96 |
| target site 6 | | |
| (PAM) | | |
| sgRNA space for | UUCUUUUACAUAUGGGUCC | 97 |
| target site 6 | | |
| sgRNA for target | GUUCUUUUACAUAUGGGUCCGUUUUAGAGCUAGAAAUA | 98 |
| site 6 | GCAAGUUAAAAUAAGGCUAGUCCGUUAUCAACUUGAAA | |
| | AAGUGGCACCGAGUCGGUGCUUUU | |
| IFNγ exon 1 | TTCTGCTTCTTTTACATAT (GGG) | 99 |
| target site 7 | | |
| (PAM) | | |
| sgRNA space for | UUCUGCUUCUUUUACAUAU | 100 |
| target site 7 | | |
| sgRNA for target | GUUCUGCUUCUUUUACAUAUGUUUUAGAGCUAGAAAUA | 101 |
| site 7 | GCAAGUUAAAAUAAGGCUAGUCCGUUAUCAACUUGAAA | |
| | AAGUGGCACCGAGUCGGUGCUUUU | |
| IFNγ exon 1 | TTTCTGCTTCTTTTACATA (TGG) | 102 |
| target site 8 | | |
| (PAM) | | |
| sgRNA space for | UUUCUGCUUCUUUUACAUA | 103 |
| target site 8 | | |
| sgRNA for target | GUUUCUGCUUCUUUUACAUAGUUUUAGAGCUAGAAAUA | 104 |
| site 8 | GCAAGUUAAAAUAAGGCUAGUCCGUUAUCAACUUGAAA | |
| | AAGUGGCACCGAGUCGGUGCUUUU | |

### EXAMPLES

### Example 1: Preparation of Genetically Engineered T Cells Expressing Bi-Specific Chimeric Antigen Receptor (CAR)

Blood samples were collected from human patient donors and the peripheral blood mononuclear cells (PBMCs) were isolated from the blood samples via routine practice. Lentiviral expression vectors coding for an anti-CD19/anti-BCMA bispecific CAR, and optionally an anti-IFNγ scFv (SEQ ID NO: 21 or SEQ ID NO: 18) and/or an anti-IL6 scFv (SEQ ID NO: 35) were introduced into the PBMCs to allow for expression of the bispecific CAR and optionally the anti-IFNγ scFv and the anti-IL6 scFv.

Two designs of the anti-CD19/anti-BCMA bispecific CAR were explored: (a) anti-CD19 VHH/anti-BCMA scFv, and (b) anti-BCMA VHH/anti-CD19 scFv. All of the bispecific CAR constructs contain the CD8 lead sequence (SEQ ID NO: 45), the GS linker (SEQ ID NO: 57), the CD8 hinge domain (SEQ ID NO: 53), the CD8 transmembrane domain (SEQ ID NO: 38), the 4-1BB co-stimulatory domain (SEQ ID NO: 39), the IL-2Rb signaling domain (SEQ ID NO: 40), and the CD3z signaling domain (SEQ ID NO: 42). See Sequence Table 2. The construct of (a) comprises the amino acid sequence of SEQ ID NO: 65; the construct of (b) comprises the amino acid sequence of SEQ ID NO: 67.

In some instances, a bicistronic expression vector comprising the coding sequence of construct (a) or (b) and the coding sequence of an anti-IFNγ scFv (SEQ ID NO: 21 or SEQ ID NO: 18) connected by a T2A-coding sequence linker was used to produce genetically engineered T cells expressing both the bi-specific CAR and the anti-IFNγ scFv (secretive). In other instances, a tricistronic expression vector comprising the coding sequence of construct (a) or (b), the coding sequence of an anti-IFNγ scFv (SEQ ID NO: 21 or SEQ ID NO: 18) connected to the coding sequence of (a) or (b) by a T2A-coding sequence, and the coding sequence of the anti-IL6 scFv (SEQ ID NO:35) connected to the coding sequence of an anti-IFNγ scFv via a P2A-coding sequence.

Primary T cells collected from healthy donors were activated by anti-CD3/CD28 beads (Thermo scientific). One day later, the T cells were transduced with the lentiviral vectors encoding one of the above-noted bi-specific CAR and optionally the anti-IFNγ scFv and the anti-IL6 scFv disclosed above. The transduced cells were expanded and tested for CD3 expression by FACS analysis and the CD3+ population was gated for further analysis.

CAR expression was analyzed by flow cytometry using a biotinylated primary antibody recognizing the antibody fragment in the CAR and a fluorescence labeled secondary antibody conjugated with Streptavidin.

Functionality of the bi-specific Car-T cells was analyzed by coculture of the CAR-T cells with target antigen-presenting cells (APCs) or target tumor cells to evaluate CAR-T cell proliferation, cytotoxicity, or a combination thereof.

### Example 2: Treating Acute Lymphocytic Leukemia (ALL) Patient with Anti-CD19/Anti-BCMA Bi-Specific CAR-T Cells

Human patients having acute lymphocytic leukemia (ALL) were treated with the bi-specific CAR-T cells as detailed below.

(A) Treatment with Bi-Specific CAR T Cells Secreting Anti-IFNγ scFv

A patient (ALL Patient 1) diagnosed with refractory and relapsed acute lymphocytic leukemia (ALL) was administered via intravenous infusion, bi-specific CAR T cells (anti-BCMA VHH/anti-CD19 scFv, see design (b) in Example 1) secreting only the exemplary anti-IFNγ scFv (see Example 1 above, comprising the amino acid sequence of SEQ ID NO: 21) at a dose of 0.4×10⁸ CAR+ T cells, after a standard lymphodepletion treatment.

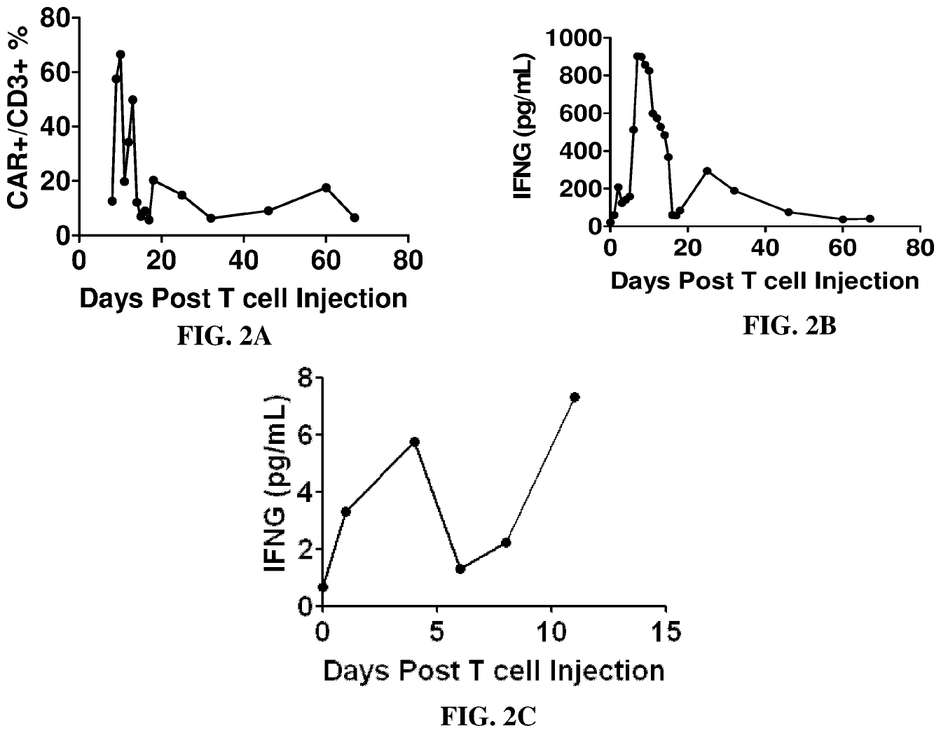

After the treatment, blood samples were collected from the patient. A significant expansion of the CAR-T cells was detected over time (FIG. 2A) and low levels of IFNγ were detected (FIG. 2B) in the blood samples. This result suggest that the bispecific anti-CD19/BCMA CAR-T cells, which co-express the anti-IFNγ scFv, are sufficient to induce durable CAR+ T cell expansion. This patient showed complete response in clinical efficacy. During this treatment, only grade 2 CRS was observed.

(B) Treatment with Bi-Specific CAR-T Cells Secreting Both Anti-IL6 scFv and Anti-IFNγ scFv

A patient (ALL Patient 2) diagnosed with ALL were administered via intravenous infusion the bi-specific CAR-T cells (anti-BCMA VHH/anti-CD19 scFv, see design (b) in Example 1) expressing both the anti-IL6 scFv and the anti-IFNγ scFv comprising the amino acid sequence of disclosed in Example 1 above. This patient showed complete response in clinical efficacy. During this treatment, only grade 1 CRS was observed. Similar to Patient 1, Patient 2 also showed low levels of IFNγ in blood samples after the treatment (FIG. 2C).

### Example 3: Treating Multiple Myeloma (MM) Patients with Bi-Specific Anti-CD19/Anti-BCMA CAR-T Cells

Up to 3 patients (MM Patient 1, MM Patient 2, and MM Patient 3) diagnosed with refractory and relapsed MM was administered CAR-T cells co-expressing the bispecific CAR construct (a) and the anti-IFNγ scFv comprising the amino acid sequence of SEQ ID NO: 21 as disclosed in Example 1 above via intravenous infusion (Patient 1, 0.4×10⁸, Patient 2, 0.8×10⁸; Patient 3, 0.8×10⁸ CAR+ T cells). One patient (MM Patient 4) diagnosed with refractory and relapsed MM was administered CAR-T cells expressing the bispecific CAR construct (a) but not the anti-IFNγ scFv.

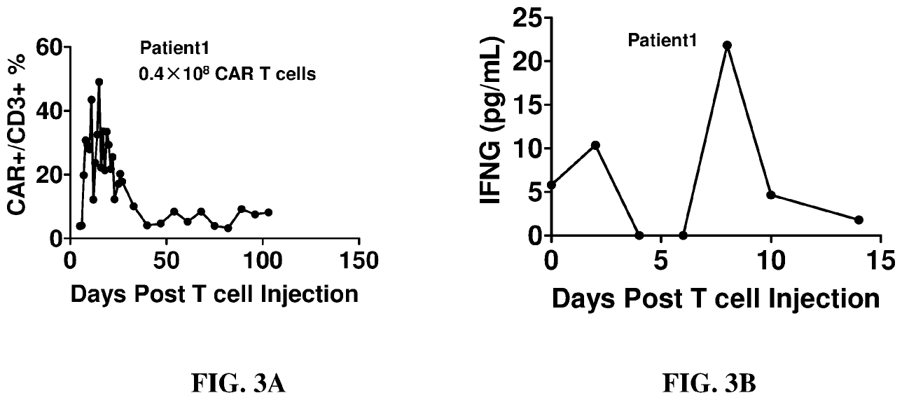

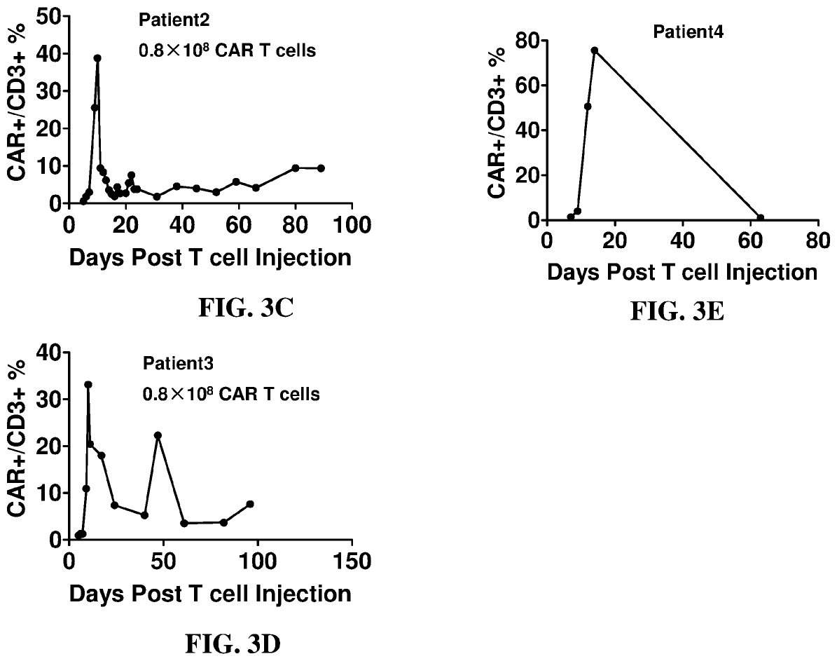

Following the treatment, CAR+ T cell expansion and levels of IFNγ were determined in each of the MM patient. Significant expansion of CAR+ T cells was detected in all of the MM patients treated in this example. FIGS. 3A and 3C-3D. Low levels of IFNγ were also detected in the peripheral blood of the patients treated with CAR-T cells expressing both the bispecific CAR and the anti-IFNγ scFv. See FIG. 3B for data from one representative patient. This indicates that the bi-specific anti-CD19/BCMA CAR-T cells co-expressing the anti-IFNγ scFv are capable of inducing robust CAR+ T cell expansion. The MM patients treated with the co-expressing the bispecific CAR and the anti-IFNγ scFv achieved complete response (CR) after the treatment. Although bone marrow examination detected 79.5% aberrant plasma cells in patient 1 before treatment, there was only transient mild hypotension during treatment successfully resolved by 10 mg of Norepinephrine in 1 day, and therefore grade 3 CRS observed. During this treatment in patient 2 and patient 3, only grade 1 CRS was observed.

CAR-T cell expansion was also observed in the MM patient treated with the T cells expressing the bi-specific CAR but not the anti-IFNγ scFv. FIG. 3E. Clinical response of this patient is under evaluation. During this treatment, only grade 1 CRS was observed.

### Example 4: In Vitro Cytotoxicity Assay of Bi-Specific CAR-T Cells

The in vitro cytotoxicity of CAR-T cells co-expressing the bispecific CAR construct (a) and the anti-IFNγ scFv disclosed in Example 1 above (SEQ ID NO: 21), and CAR-T cells expressing only the bispecific CAR construct (a) was evaluated in this example.

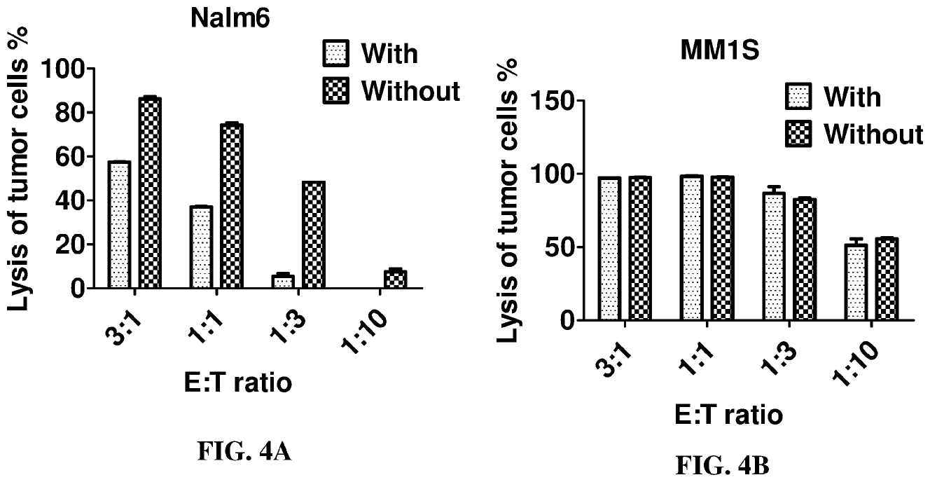

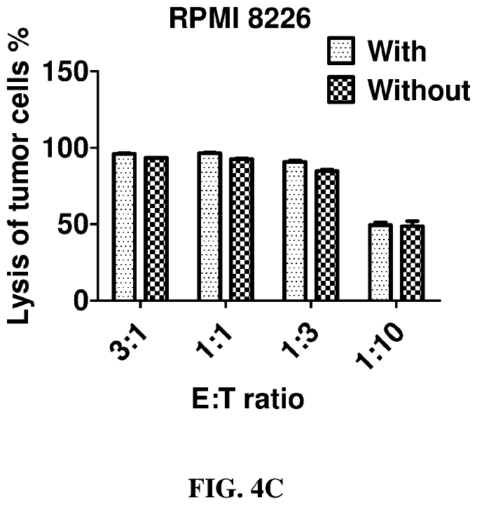

Human T cells were activated and transduced to generate genetically engineered T cells expressing both the bi-specific CAR and the anti-IFNγ scFv, or only the bi-specific CAR. The resulting engineered T cells were incubated with target tumor cells expressing green fluorescent protein (GFP, as a reporter) at various effector to target (E:T) ratios. Killing efficacy was assessed by flow cytometry by counting the number of live GFP+ target cells, which is in inverse correlation to the level of cytotoxicity. As shown in FIGS. 4A-4C, both types of CAR-T cells showed certain levels of cytotoxicity against Nalm6 cells (B cell precursor leukemia cells), MM1S cells (multiple myeloma cells), and RPMI 8226 cells (plasmacytoma cells). Co-expression of the anti-IFNγ scFv did not show significant impact on the CAR-T cell cytotoxicity against the MM1S cells and RPMI 8226 cells; however, it was found to reduce the cytotoxicity against Nalm6 cells. See FIG. 4A relative to FIGS. 4B and 4C.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A bi-specific chimeric antigen receptor (CAR) polypeptide, comprising:
a) a first antigen binding moiety,
b) a second antigen binding moiety,
c) a co-stimulatory signaling domain, and
d) a cytoplasmic signaling domain,
wherein the first antigen binding moiety is a single domain antibody variable fragment (VHH) and the second antigen binding moiety is a single chain variable fragment (scFv), and
wherein the first antigen binding moiety binds a first tumor-associated antigen, and the second antigen binding moiety binds a second tumor-associated antigen, which optionally is different from the first tumor associated antigen.

2. The bi-specific CAR polypeptide of claim 1, wherein the first and second tumor antigens are selected from the group consisting of 5T4, CD2, CD3, CD5, CD7, CD19, CD20, CD22, CD30, CD33, CD38, CD70, CD123, CD133, CD171, CEA, CS1, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, Claudin 18.2, and VEGFRII.

3. The bi-specific CAR polypeptide of claim 1, wherein the first tumor antigen is CD19 and the second tumor antigen is BCMA, or vice versa.

4. The bi-specific CAR polypeptide of claim 3, wherein the first antigen binding moiety is a VHH fragment binding to CD19 (anti-CD19 VHH) and the second antigen binding moiety is a scFv binding to BCMA (anti-BCMA scFv); or wherein the first antigen binding moiety is a VHH binding to BCMA (anti-BCMA VHH) and the second antigen binding moiety is a scFv fragmenting binding to CD19 (anti-CD19 scFv).

5. The bi-specific CAR polypeptide of claim 4, wherein the anti-CD19 scFv comprises the amino acid sequence of SEQ ID. NO: 7, 8, or 9; and/or wherein the anti-BCMA VHH comprises the amino acid sequence of SEQ ID NO: 4, 5, or 6.

6. The bi-specific CAR polypeptide of claim 5, wherein a) and b) comprises the amino acid sequence of SEQ ID NO:11, 44, 63, 64, 67, 68, 71, or 79, optionally SEQ ID NO: 11.

7. The bi-specific CAR polypeptide of claim 4, wherein the anti-CD19 VHH comprises the amino acid sequence of SEQ ID NO: 1, 2, or 3; and/or wherein the anti-BCMA scFv comprises the amino acid sequence of SEQ ID NO: 10.

8. The bi-specific CAR polypeptide of claim 7, wherein a) and b) comprises the amino acid sequence of SEQ ID NO: 12, 65, 66, 69, 70, 72, 78, or 80, optionally SEQ ID NO: 12.

9. A bi-specific chimeric antigen receptor (CAR) polypeptide, comprising:
a) a first antigen binding moiety, which is a truncated fragment of APRIL that binds to BCMA;
b) a second antigen binding moiety, which is a single domain antibody variable fragment (VHH) or a single chain variable fragment (scFv) that binds a tumor associated antigen,
c) a co-stimulatory signaling domain, and
d) a cytoplasmic signaling domain,

10. The bi-specific CAR polypeptide of claim 9, wherein the truncated fragment of APRIL that binds BCMA comprises an amino acid sequence at least 90% identical to SEQ ID NO: 58; optionally wherein the truncated fragment of APRIL comprises the amino acid sequence of SEQ ID NO: 58.

11. The bi-specific CAR polypeptide of claim 9, wherein the second antigen-binding moiety is an anti-CD19 scFv or an anti-CD19 VHH.

12. The bi-specific CAR polypeptide of claim 11, wherein the anti-CD19 scFv comprises the amino acid sequence of SEQ ID NO: 7, 8, or 9; or wherein the anti-CD19 VHH comprises the amino acid sequence of SEQ ID NO: 1, 2, or 3.

13. The bi-specific CAR polypeptide of claim 12, wherein a) and b) comprise the amino acid sequence of SEQ ID NO: 59, 60, 61, or 62.

14. The bi-specific CAR polypeptide of claim 1, further comprising a peptide linker between the first antigen binding moiety and the second antigen binding moiety, optionally wherein the peptide linker is about 4-40 amino acids in length.

15. The bi-specific CAR polypeptide of claim 1, wherein the co-stimulatory signaling domain is from 4-1BB or CD28.

16. The bi-specific CAR polypeptide of claim 1, wherein the cytoplasmic signaling domain comprises a CD3 z cytoplasmic signaling domain, an IL-2Rβ cytoplasmic signaling domain, or a combination thereof.

17. The bi-specific CAR polypeptide of claim 16, wherein the cytoplasmic signaling domain comprises the CD3 z cytoplasmic signaling domain, which optionally comprises a STAT binding motif.

18. The bi-specific CAR polypeptide of claim 1, further comprising a transmembrane domain, a hinge domain, or a combination thereof, which optionally is located between the first or second antigen binding moiety and the co-stimulatory domain.

19. The bi-specific CAR polypeptide of claim 18, wherein the transmembrane domain and/or the hinge domain is from CD8.

20. The bi-specific CAR polypeptide of claim 1, which comprises the amino acid sequence of any one of SEQ ID NOs: 63-70.

21. A population of genetically engineered immune cells, which expressing a bi-specific CAR polypeptide of claim 1.

22. The population of genetically engineered immune cells of claim 21, which further comprise one or more of the following features:
e) have one or more disrupted endogenous genes encoding one or more proinflammatory cytokines; and
f) express one or more antagonists targeting the proinflammatory cytokines.

23. The population of genetically engineered immune cells of claim 22, wherein the proinflammatory cytokines are selected from the group consisting of interferon gamma (IFNγ), interleukin 6 (IL-6), GM-CSF, and interleukin 1 (IL-1).

24. The population of genetically engineered immune cells of claim 22, wherein the genetically engineered immune cells comprise a disrupted endogenous interferon gamma gene, a disrupted endogenous GM-CSF gene, or a combination thereof.

25. The population of genetically engineered immune cells of claim 24, wherein the endogenous interferon gamma gene, the endogenous GM-CSF gene, or both are disrupted by a CRISPR/Cas gene editing system.

26. The population of genetically engineered immune cells of claim 22, wherein the genetically engineered immune cells express an IL-6 antagonist, an IFNγ antagonist, an IL-1 antagonist, or a combination thereof.

27. The population of genetically engineered immune cells of claim 26, wherein the IL-6 antagonist is an antibody specific to human IL6 (anti-IL6 antibody) or an antibody specific to human IL6R (anti-IL6R antibody), and/or wherein the IFNγ antagonist is an antibody specific to human IFNγ (anti-IFNγ antibody).

28. The population of genetically engineered immune cells of claim 27, wherein the anti-IL6 antibody, the anti-IFNγ antibody, or both are scFv antibodies.

29. The population of genetically engineered immune cells of claim 28, wherein the genetically engineered immune cells express an anti-IFNγ scFv comprising:
(i) a heavy chain variable region, which comprises the amino acid sequence of SEQ ID NO: 13, and a light chain variable region, which comprises the amino acid sequence of SEQ ID NO: 14;
(ii) a heavy chain variable region, which comprises the amino acid sequence of SEQ ID NO: 16, and a light chain variable region, which comprises the amino acid sequence of SEQ ID NO: 17; or
(iii) a heavy chain variable region, which comprises the amino acid sequence of SEQ ID NO: 19, and a light chain variable region, which comprises the amino acid sequence of SEQ ID NO: 20.

30. The population of genetically engineered immune cells of claim 29, wherein the anti-IFNγ scFv comprises the amino acid sequence of SEQ. ID. NO: 15, 18, or 21.

31. The population of genetically engineered immune cells of claim 30, wherein the genetically engineered immune cells express a bi-specific CAR comprising the amino acid sequence of any one of SEQ ID NOs: 44, 63-70 or 78-80.

32. The population of genetically engineered immune cells of claim 28, wherein the genetically engineered immune cells express an anti-IL6 scFv comprising:
(a) a heavy chain variable region, which comprises the amino acid sequence of SEQ ID NO: 24, and a light chain variable region, which comprises the amino acid sequence of SEQ ID NO: 25;
(b) a heavy chain variable region, which comprises the amino acid sequence of SEQ ID NO: 26, and a light chain variable region, which comprises the amino acid sequence of SEQ ID NO: 27; or
(c) a heavy chain variable region, which comprises the amino acid sequence of SEQ ID NO: 30, and a light chain variable region, which comprises the amino acid sequence of SEQ ID NO: 31.

33. The population of genetically engineered immune cells of claim 28, wherein the genetically engineered immune cells express an anti-IL6R scFv comprising:
(a) a heavy chain variable region, which comprises the amino acid sequence of SEQ ID NO: 22, and a light chain variable region, which comprises the amino acid sequence of SEQ ID NO: 23;
(b) a heavy chain variable region, which comprises the amino acid sequence of SEQ ID NO: 28, and a light chain variable region, which comprises the amino acid sequence of SEQ ID NO: 29; or
(c) a heavy chain variable region, which comprises the amino acid sequence of SEQ ID NO: 32, and a light chain variable region, which comprises the amino acid sequence of SEQ ID NO: 33.

34. The population of genetically engineered immune cells of claim 32, wherein the anti-IL6 scFv or anti-IL6R scFv comprises the amino acid sequence of SEQ ID NO: 34, 35, 36, or 37.

35. The population of genetically engineered immune cells of claim 22, wherein genetically engineered immune cells express an IL-1 antagonist, and wherein the IL-1 antagonist is IL-1RA, which comprises the amino acid sequence of SEQ ID NO: 54.

36. The population of genetically engineered immune cells of claim 22, wherein the genetically engineered immune cells comprise T cells, tumor infiltrating lymphocytes, Natural Killer (NK) cells, dendritic cells, macrographs, B cells, neutrophils, eosinophils, basophils, mast cells, myeloid-derived suppressor cells, mesenchymal stem cells, precursors thereof, or a combination thereof.

37. The population of genetically engineered immune cells of claim 22, wherein the immune cells are human immune cells.

38. The population of genetically engineered immune cells of claim 37, which comprise human T cells.

39. A pharmaceutical composition, comprising a population of immune cells of claim 22 and a pharmaceutically acceptable carrier.

40. A method for reducing or eliminating undesired cells in a subject, the method comprising administering to a subject in need thereof a therapeutically effective amount of the population of immune cells of claim 22 or a pharmaceutical composition comprising the population of immune cells of claim 39.

41. The method of claim 40, wherein the subject is a human patient having a cancer, which comprises cancer cells expressing the first tumor associated antigen, the second tumor associated antigen, or both.

42. The method of claim 40, wherein the subject is a human patient having a solid tumor or a hematological cancer.

43. The method of claim 42, wherein the human patient has a solid tumor, which is selected from the group consisting of breast cancer, lung cancer, pancreatic cancer, liver cancer, glioblastoma (GBM), prostate cancer, ovarian cancer, mesothelioma, colon cancer, and stomach cancer.

44. The method of claim 42, wherein the human patient has a hematological cancer, which is leukemia, lymphoma, or multiple myeloma.
